(19) United States
(12) Patent Application Publication
(10) Pub. No.: US 2025/0257364 A1
(43) Pub. Date: Aug. 14, 2025

(54) RNA-GUIDED DNA TRANSPOSITION

(71) Applicant: Wisconsin Alumni Research Foundation, Madison, WI (US)

(72) Inventors: Jason Peters, Madison, WI (US); Amy Banta, Madison, WI (US)

(73) Assignee: Wisconsin Alumni Research Foundation, Madison, WI (US)

(21) Appl. No.: 19/050,317

(22) Filed: Feb. 11, 2025

Related U.S. Application Data
(60) Provisional application No. 63/552,266, filed on Feb. 12, 2024.

(51) Int. Cl. C12N 15/78; C12N 9/12; C12N 9/22; C12N 15/11; C12N 15/70

(52) U.S. Cl. CPC C12N 15/78; C12N 9/1241; C12N 9/22; C12N 15/111; C12N 15/70; C12N 2310/20; C12Y 207/07

(57) ABSTRACT

Described herein is a system for RNA-guided DNA transposition in a bacterial cell's genome including a) a first non-replicating plasmid expressing an engineered Clustered Regularly Interspaced Short Palindromic Repeats associated (Cas) (CRISPR-Cas) protein and a transposase; and b) a second non-replicating donor plasmid, the second non-replicating donor plasmid including an antibiotic selection marker, a guide RNA (gRNA) sequence and a cargo sequence flanked by a right transposon end sequence and a left transposon end sequence, wherein the gRNA is specific for a target site, and wherein the cargo sequence comprises a promoter, a protein expression sequence, or a combination thereof. The first non-replicating plasmid and the second non-replicating donor plasmid, when introduced into a cell, provide insertion of the cargo sequence into the bacterial cell's genome as directed by the gRNA.

### CROSS-REFERENCE TO RELATED APPLICATIONS

This application claims priority to U.S. Provisional Application 63/552,266 filed on Feb. 12, 2024, which is incorporated herein by reference in its entirety.

### STATEMENT REGARDING FEDERALLY SPONSORED RESEARCH & DEVELOPMENT

This invention was made with government support under DE-SC0018409 awarded by the US Department of Energy. The government has certain rights in the invention.

### SEQUENCE LISTING

The Instant Application contains a Sequence Listing which has been submitted electronically in XML format and is hereby incorporated by reference in its entirety. Said XML copy, created on Jan. 17, 2025, is named “SEQ_LIST—107668305-P230410US02.xml” and is 86,503 bytes in size. The Sequence Listing does not go beyond the disclosure in the application as filed.

### FIELD OF THE DISCLOSURE

The present disclosure is related to a system and methods for RNA-guided DNA transposition in a bacterial cell's genome. The methods provide for insertion of cargo sequences such as promoters for overexpression of bacterial genes.

### BACKGROUND

Overexpression (OE) studies have been invaluable for gene phenotyping in medical, industrial, and basic contexts. When used in functional genomics, OE enables functional interrogation of genes that are normally cryptic under screening conditions, mimics natural resistance mechanisms, and complements loss of function approaches by identifying genes with oppositely signed phenotypes when knocked down versus overexpressed. Clustered Regularly Interspaced Short Palindromic Repeats activation (CRISPRa) has allowed routine, genome-scale overexpression screening in eukaryotes for drug synergy/antagonism or sensitivity/resistance to toxic plant compounds that reduce bioproduct yields. Such screens have been highly effective at identifying direct targets and pathways that mitigate or potentiate drug function (Jost, M. & Weissman, J. S. CRISPR Approaches to Small Molecule Target Identification. ACS Chem Biol 13, 366-375 (2018); Lian, J., Schultz, C., Cao, M., HamediRad, M. & Zhao, H. Multi-functional genome-wide CRISPR system for high throughput genotype-phenotype mapping. Nat Commun 10, 5794 (2019)). For example, a recent screen in mammalian cells for targets of the anti-cancer drug, rigosertib, leveraged genome-scale CRISPR interference (CRISPRi) knockdowns with CRISPRa to identify target pathways that showed oppositely signed phenotypes. OE has also enhanced strain optimization for industrial purposes, such as a genome-wide CRISPRa screen in Saccharomyces cerevisiae for genes involved in resistance to the biofuel production stressor, furfural.

Existing genome-scale OE approaches in bacteria are typically not targeted, systematic, or practical. Approaches that rely on random integration of promoter containing transposons to overexpress downstream genes are untargeted and require hyper-saturated transposon (Tn)libraries to obtain insertions in intergenic regions that do not disrupt adjacent genes. Open Reading Frame (ORF) libraries cloned into plasmids are technically targeted approaches, but are tedious and expensive to construct, limiting their utility in non-model bacteria. Further, the use of plasmids presents additional complications, such as the constant requirement for antibiotic selection for maintenance (complicating antibiotic function screens), copy number variation that increases experimental noise, and the inability to overexpress protein complexes that are typically found in operons in bacterial genomes. An innovative new OE technique, “Dub-seq” uses genomic fragments cloned into multi-copy plasmids that can be quantified by barcode sequencing; however, this approach is also not targeted, shares some downsides with other plasmid-based approaches, and is constrained in capturing protein complex phenotypes depending on the size of the cloned genomic fragments.

CRISPRa in eukaryotes uses a catalytically inactive Cas9 protein (dCas9) and single guide RNAs (sgRNAs) to deliver activator proteins to promoters; these activators open chromatin at the promoter, enhancing transcription activation. In contrast to other methods, CRISPRa-based OE in bacteria requires direct interactions between activator proteins and RNA polymerase (RNAP) that dictate stringent activator-RNAP spacing and orientation on DNA. Recent improvements to bacterial CRISPRa using engineered transcription factors partially mitigated the spacing requirements and increased overall activation; however, even in these optimized systems, less than half of targeted endogenous genes could be upregulated and individual guides showed substantial variation in activity. The cause(s) of these efficacy issues are largely unknown.

Naturally occurring CRISPR-associated transposon (CAST) systems (Peters, J. E., Makarova, K. S., Shmakov, S. & Koonin, E. V. Recruitment of CRISPR-Cas systems by Tn7-like transposons. PNAS 114, E7358-E7366 (2017)) have been engineered to generate targeted insertions in bacterial genomes with high efficiency. Although several CAST systems have been described, the Tn6677 system from Vibrio cholerae (Vc CAST) is among the best characterized. In Vc CAST, a nuclease-deficient Type I-F CRISPR system is paired with a Tn7-like transposase to achieve guide RNA-directed transposition. Binding of a Vc CAST-gRNA complex spacer to a matching protospacer results in transposition of DNA flanked by Tn6677 ends approximately 49 bp downstream of the protospacer. Transposon DNA can be inserted in two orientations with respect to the Tn6677 ends (R-L or L-R) but is highly biased toward the R-L orientation (Vo, P. L. H. et al. CRISPR RNA-guided integrases for high-efficiency, multiplexed bacterial genome engineering. Nat Biotechnol 39, 480-489 (2021)). Vc CAST is highly precise, with nearly all tested guides showing >95% on-target insertion. The utility of Vc CAST has been demonstrated for high efficiency editing and even targeting individual strains in a microbiome, but the system has not been optimized for functional genomics. Moreover, the use of Tn6677 to deliver regulatory elements—such as promoters—has been underexplored.

What is needed are novel targeted overexpression systems for bacteria.

### BRIEF SUMMARY

In an aspect, a system for RNA-guided DNA transposition in a bacterial cell's genome comprises a) a first non-replicating plasmid expressing an engineered Clustered Regularly Interspaced Short Palindromic Repeats associated (Cas) (CRISPR-Cas) protein and a transposase; and b) a second non-replicating donor plasmid, the second non-replicating donor plasmid comprising an antibiotic selection marker, a guide RNA (gRNA) sequence and a cargo sequence flanked by a right transposon end sequence and a left transposon end sequence, wherein the gRNA is specific for a target site, and wherein the cargo sequence comprises a promoter, a protein expression sequence, or a combination thereof, wherein the first non-replicating plasmid and the second non-replicating donor plasmid, when introduced into a cell, provide insertion of the cargo sequence into the bacterial cell's genome as directed by the gRNA.

In another aspect, a method of RNA-guided DNA transposition in a bacterial cell's genome comprises transferring the first and second non-replicating plasmids described above into the bacterial cell, providing insertion of the cargo sequence into the bacterial cell's genome as directed by the gRNA.

### BRIEF DESCRIPTION OF THE DRAWINGS

FIGS. 1a-e illustrate a mobilizable, selectable, dual-plasmid system for CRISPR-guided transposition. 1a. Schematic of a targeted transposition gene knockout (CRISPRt) and gene overexpression (CRISPRtOE) system optimized for functional genomics. 1b. Schematic of mechanism of Vibrio cholera Type I-F CRISPR-associated transposon (Vc CAST) system. 1c. Schematic of strain construction using the CRISPRt targeted transposition system. Plasmids have pir-dependent origins of replication preventing replication in the bacterial recipient. One plasmid encodes the minimal Type I-F CRISPR-Cas (Cas678) and Tn7-like transposase (TnsABC and TniQ) machinery, and a second plasmid contains a transposon carrying the guide RNA and antibiotic resistance expression cassettes. These plasmids are transferred by co-conjugation to a recipient bacterium by E. coli donor strains with a chromosomal copy of the RP4 transfer machinery. Inside the recipient cell, the transposon (flanked in yellow) is inserted onto the chromosome at a site determined by the sequence of the guide RNA. Selection on antibiotic plates lacking diaminopimelic acid (DAP) selects for transconjugants and against the E. coli donor strains. 1d. Specificity of CRISPRt disruption of gfp and lacZ in E. coli measured by Tnseq. Mapping of location of transposon insertion sites to the E. coli genome after CRISPRt targeted transposition with gfp1 guide (226,671 reads) or LZ1 guide (216,801 reads). 1e. Insertion position (bp downstream of protospacer) frequency of CRISPRt disruption of gfp and lacZ in E. coli (RL orientation). Percentage of on-target, RL orientation insertions for all positions with >0.01%.

FIGS. 2a-c show transcription impacts efficacy of CRISPRt guides. 2a. Schematic of guides in pooled mScarlet-I disruption experiment. Twenty-three guides matching protospacers located on top (T1-24) or bottom (B1-24) strand of the mScarlet-I encoding gene with a range of CN guides (Table 2). 2b. Relative efficacy of CRISPRt mScarlet-I guides in a pooled screen targeting mScarlet-I expressed from a transposon in the attTn7 site on the E. coli chromosome. Relative efficacy is expressed as the log₂-fold change of the frequency in the CRISPRt libraries (sJMP10519 and sJMP10520) vs. the frequency in the original plasmid construct libraries (sJMP10505 and sJMP10506). Average and standard deviation of two separate CRISPRt libraries (gentamicin^{R} and kanamycin^{R}) containing all 46 guides. 2c. Absolute transposition efficiency of 3 individual CRISPRt guides (T03, T09, T21) targeting mScarlet-I cassettes in the E. coli attTn7 site either with a promoter (+transcription) or without a promoter (-transcription) measured by plating efficiency with and without selection (+/−kanamycin) in triplicate. Error represents the average of n=3 assays.

FIGS. 3a-c show tunable overexpression of chromosomally located genes using CRISPRtOE. 3a. Schematic of CRISPRtOE targeted overexpression system. The chromosomal target is an mScarlet-I gene preceded by a ‘Landing Pad’ (LP) with no promoter, a PAM, and the LZ1 protospacer. The CRISPRtOE construct has a transposon carrying guide RNA and antibiotic resistance expression cassettes and an outward facing promoter. Co-conjugation of strains carrying the CRISPRtOE construct and the CRISPRt-H plasmid (harboring the Vc CAST machinery) with a promoterless mScarlet-I reporter recipient strain results in a CRISPRtOE strain. 3b. mScarlet-I fluorescence analysis of E. coli CRISPRtOE isolates (no promoter or synthetic promoters A, V, and H) compared to the parent (promoterless mScarlet-1) strain. 3c. Fold effect of CRISPRtOE mScarlet-I overexpression using Promoter H in eight diverse Alpha- and Gammaprotebacteria (E. coli (Eco), Enterobacter cloacae (Ecl), Klebsiella pneumoniae (Kpn), Acinetobacter baumannii (Aba), Pseudomonas aeruginosa (Pae), Pseudomonas putida (Ppu), Zymomonas mobilis (Zmo), and Shewanella oneidensis (Son)). CRISPRtOE transposon insertion position and fluorescence measurements were determined for twelve isolates. Fluorescence measurements were normalized to cell density (OD₆₀₀). Values are shown for isolates with on-target, RL orientation CRISPRtOE insertions and fold changes for on-target and RL orientation CRISPRtOE isolates compared to a strain with promoterless mScarlet-I. Error is expressed for the median value of 8-12 isolates in n=3 (Eco and Zmo) or n=2 (Ecl, Kpn, Aba, Pae, Ppu, and Son) assays.

FIGS. 4a-c show CRISPRtOE informs antibiotic mode of action. 4a. Schematic of CRISPRtOE competitive growth assay. A pooled CRISPRtOE strain library is constructed by amplification of spacer sequences from a pooled oligo library and transfer onto the chromosome of a recipient strain. Insertion of the CRISPRtOE transposon is either upstream of genes of interest or within a control gene (e.g. lacZ) in E. coli. A CRISPRtOE pooled library screen is conducted by culturing a CRISPRtOE pooled library and control library for multiple generations in the presence or absence of a chemical or condition of interest. The change in the composition of the pooled library before and after treatment is measured by amplification and NGS sequencing of the spacer sequences. 4b. CRISPRtOE pooled library screen. Fitness of pooled strains overexpressing folA compared to the lacZ control strains in the presence of trimethoprim (TMP). 4c. MIC broth microdilution assay of individual CRISPRtOE folA strains compared to WT, a CRISPRtOE lacZ isolate, and the CRISPRtOE folA pooled library in the presence of TMP (0.04-20 μg/ml). Error is expressed for 3 separate assays.

FIGS. 5a-e show genome-scale CRISPRtOE. 5a. Genome plot showing positions of CRISPRtOE insertions in TMP-treated cells. Data are scaled to the maximum number of counts. 5b. Positions of CRISPRtOE insertions upstream of all genes in the genome. Insertion site distance d was calculated based on the distance in bp from the 3′ end of the Tn to the 5′ end of the gene. 5c. CRISPRtOE insertions into the lac locus. The scale is capped at 20 reads for visual clarity. 5d. Volcano plot of gRNA spacer counts in TMP vs DMSO control. 5e. Top positive gene hits in TMP treatment. Genes must have at least a 4-fold change in the promoter H data and show a 4-fold difference in a comparison between promoter H and no promoter to be considered. Error bars represent standard deviation (SD).

The above-described and other features will be appreciated and understood by those skilled in the art from the following detailed description, drawings, and appended claims.

### DETAILED DESCRIPTION

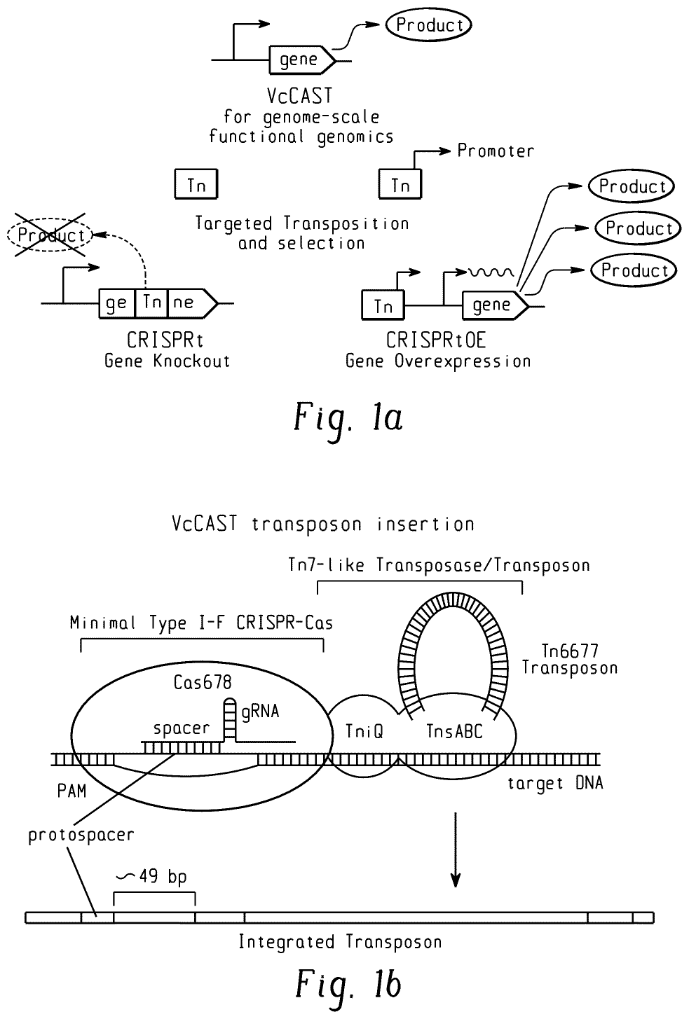

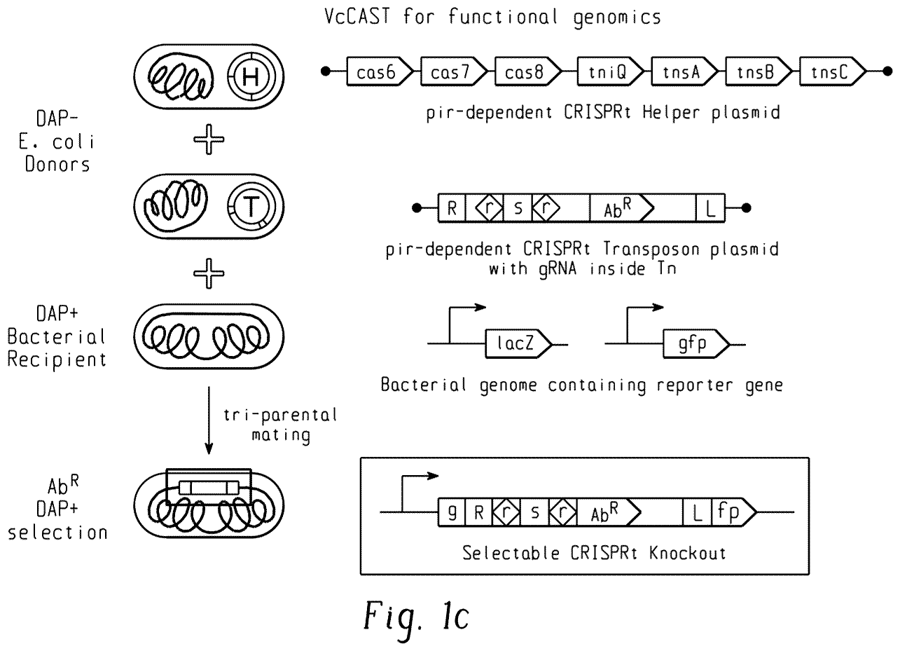

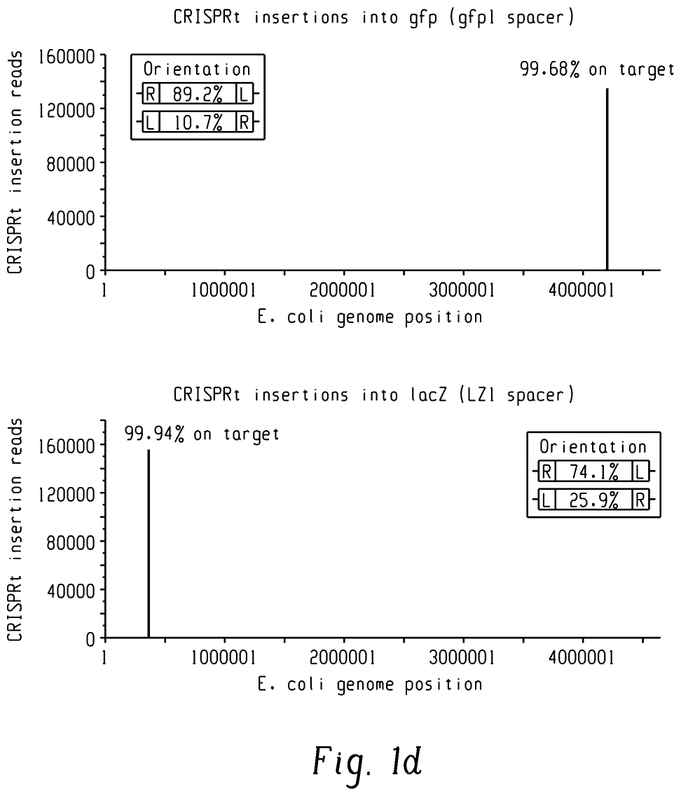

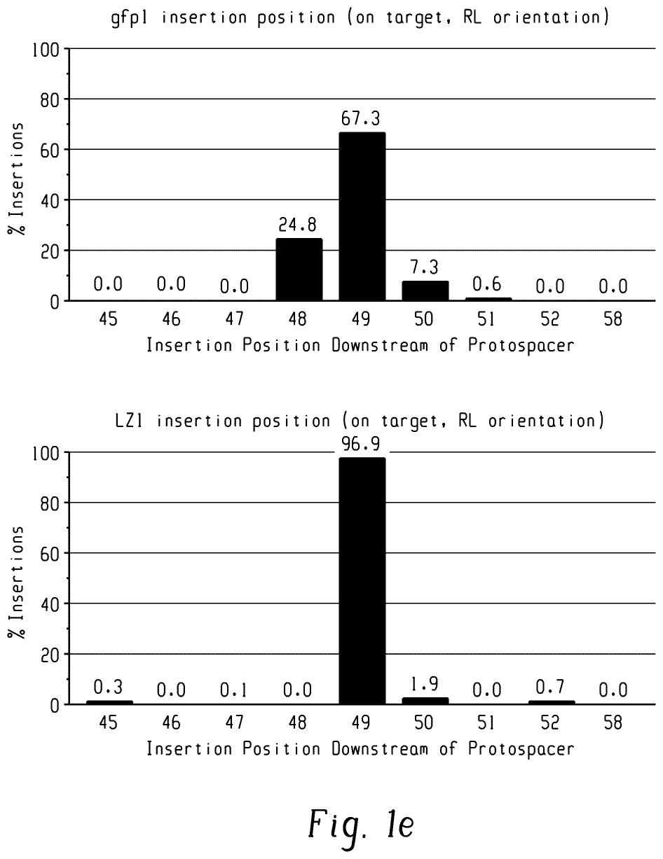

Described herein is the modification of Vc CAST for functional genomics, creating a targeted overexpression system for bacteria referred to herein as, “CRISPRtOE” (CRISPR transposition and OverExpression). CRISPRtOE can deliver a transposon with a cargo and an outward facing promoter upstream of genes to facilitate overexpression (FIG. 1a). CRISPRtOE obviates issues such as spacing between and “activation potential” of promoters found in bacterial CRISPRa. The utility of CRISPRtOE in medically and industrially-relevant bacterial species and at the genome-scale to characterize antibiotic action and resistance is demonstrated herein. In addition, CRISPRtOE can be used to deliver cargo other than promoters such as protein expression sequences, fluorescent protein encoding genes (e.g., GFP) that can be used to mark strains, biosynthetic pathways for producing natural products, catabolic pathways to enable/improve carbon source utilization, additional CRISPR systems for pathway control and characterization (e.g., CRISPR interference).

In an aspect, a system for RNA-guided DNA transposition in a bacterial cell's genome comprises a) a first non-replicating plasmid expressing an engineered Clustered Regularly Interspaced Short Palindromic Repeats associated (Cas) (CRISPR-Cas) protein and a transposase; and b) a second non-replicating donor plasmid, the second non-replicating donor plasmid comprising an antibiotic selection marker, a guide RNA (gRNA) sequence and a cargo sequence flanked by a right transposon end sequence and a left transposon end sequence, wherein the gRNA is specific for a target site, and wherein the cargo sequence comprises a promoter, a protein expression sequence, or a combination thereof. The first non-replicating plasmid and the second non-replicating donor plasmid, when introduced into a cell, provide insertion of the cargo sequence into the bacterial cell's genome as directed by the gRNA.

Advantageously, the system and methods described herein provide selectable transposon insertion; no replication of the plasmid carrying the CRISPR-Cas/transposase is required; no exogenous Cas or plasmid components remain in the recipient bacteria; and the separation of the transposase/cas and transposon elements across two plasmids provides easy modification of the cargo sequence. It was also unexpectedly found that targeting non-transcribed DNA improves the efficiency of the systems and methods described herein.

The first non-replicating plasmid expresses an engineered Clustered Regularly Interspaced Short Palindromic Repeats associated (Cas) (CRISPR-Cas) protein and a transposase.

Cas9 is a dual RNA-guided DNA endonuclease enzyme associated with the Clustered Regularly Interspaced Short Palindromic Repeats (CRISPR) adaptive immune system in Streptococcus pyogenes. dCas9 refers to Cas9 variants without endonuclease activity, which are used in CRISPR systems along with gRNAs to target specific genes or nucleotides complementary to the gRNA with PAM sequences that allow the dCas9 to bind. Although dCas9 lacks endonuclease activity, it is still capable of binding to its guide RNA and the DNA strand that is being targeted.

In an aspect, the CRISPR-Cas system is a Class 1 CRISPR-Cas system. In some cases, a Class I CRISPR-Cas system comprises Cascade (a multimeric complex consisting of three to five proteins that processes crRNA arrays), Cas3 (a protein with nuclease, helicase, and exonuclease activity that is responsible for degradation of the target DNA), and crRNA (stabilizes Cascade complex and directs Cascade and Cas3 to DNA target). A Class 1 CRISPR-Cas system may be of a subtype, e.g., Type I-A, Type I-B, Type I-C, Type I-D, Type I-E, Type I-F, Type I-U, Type III-A, Type III-B, Type-III-C, Type-III-D, or Type-IV CRISPR-Cas system.

A Class 1 Type I-A CRISPR-Cas system may comprise Cas7 (Csa2), Cas8a1 (Csx13), Cas8a2 (Csx9), Cas5, Csa5, Cas6a, Cas3′ and/or Cas3. A Type I-B CRISPR-Cas system may comprise Cas6b, Cas8b (Csh1), Cas7 (Csh2) and/or Cas5. A Type I-C CRISPR-Cas system may comprise Cas5d, Cas8c (Csd1), and/or Cas7 (Csd2). A Type I-D CRISPR-Cas system may comprise Cas10d (Csc3), Csc2, Csc1, and/or Cas6d. A Type I-E CRISPR-Cas system may comprise Cse1 (CasA), Cse2 (CasB), Cas7 (CasC), Cas5 (CasD) and/or Cas6e (CasE).

In some embodiments, the Cas protein comprises Cas5, Cas6, Cas7, and Cas8. In some embodiments, the Cas protein is derived from a Type I CRISPR-Cas system. In some embodiments, the Cas protein comprises Cas5, Cas6, Cas7, and Cas8. In some embodiments, the Type I CRISPR-Cas system is Type I-B or Type I-F. In some embodiments, the Type I CRISPR-Cas system is a Type I-F variant where the Cas8 and the Cas5 form a Cas8-Cas5 fusion.

In some embodiments, the Type I CRISPR-Cas system is a Type I-F variant. In some embodiments, the Type I-F variant is from a bacteria selected from the group consisting of: Vibrio cholerae, Photobacterium iliopiscarium, Pseudoalteromonas sp. P1-25, Pseudoalteromonas ruthenica, Photobacterium ganghwense, Shewanella sp. UCD-KL21, Vibrio diazotrophicus, Vibrio sp. 16, Vibrio sp. F12, Vibrio splendidus, Aliivibrio wodanis, and Parashewanella spongiae. In certain embodiments, the Type I-F variant is from a bacteria selected from the group consisting of Vibrio cholerae strain 4874, Photobacterium iliopiscarium strain NCIMB, Pseudoalteromonas sp. P1-25, Pseudoalteromonas ruthenica strain S3245, Photobacterium ganghwense strain JCM, Shewanella sp. UCD-KL21, Vibrio cholerae strain OYP7GO4, Vibrio cholerae strain M1517, Vibrio diazotrophicus strain 60.6 F, Vibrio sp. 16, Vibrio sp. F12, Vibrio splendidus strain UCD-SED10, Aliivibrio wodanis 06/09/160, and Parashewanella spongiae strain HJ039. In some embodiments, the Type I-F variant is from Vibrio cholera strain HE-45.

In some embodiments, the Cas protein of the CRISPR-Cas system may be derived from a Type V CRISPR-Cas system. In certain embodiments, the Cas protein may be a Cas protein of a Class 2, Type V CRISPR-Cas system (a Type V Cas protein). The Type V Cas protein may be a Type V-K Cas protein. The Cas protein may comprise an activation mutation. In one example embodiment, the Cas12k is Scytonema hofmanni Cas12k (ShCas12k). For example, the Scytonema hofmanni may be Scytonema hofmanni (UTEX B 2349). In certain example embodiments, the Cas12k is Anabaena cylindric Cas12k (AcCas12k). For example, the Anabaena cylindrica may be Anabaena cylindrica (PCC 7122).

In certain example embodiments, the CRISPR-Cas system may be a Class 2 CRISPR-Cas system. A Class 2 CRISPR-Cas system may be of a subtype, e.g., Type II-A, Type II-B, Type II-C, Type V-A, Type V-B, Type V-C, Type V-U, Type VI-A, Type VI-B, or Type VI-C CRISPR-Cas system. In some embodiments, the at least one Cas protein of the CRISPR-Cas system is derived from a Type II-A CRISPR-Cas system. In some embodiments, the at least one Cas protein is Cas9.

In an aspect, the engineered CRISPR-Cas system comprises Cas5, Cas6, Cas7 and Cas8, specifically a Type I-F variant where the Cas8 and Cas5 form a Cas8-Cas5 fusion; a Type V CRISPR-Cas system; or a Class 2 CRISPR-Cas system.

Cas proteins include SEQ ID NO: 1 (Cas8), SEQ ID NO: 2 (Cas7), and SEQ ID NO: 3 (Cas6). U.S. Ser. No. 10/947,534 is incorporated herein by reference for its disclosure of Cas polynucleotide and polypeptide sequences.

The amino acid sequence of Cas8 (Cas5/Cas8) may comprise SEQ ID NO: 1 or an equivalent thereof. The amino acid sequence of Cas8 (Cas5/Cas8) may comprise an amino acid sequence about 80% to about 100%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, or about 100%, identical to the amino acid sequence set forth in SEQ ID NO: 1.

The amino acid sequence of Cas7 may comprise SEQ ID NO: 2 or an equivalent thereof. The amino acid sequence of Cas7 may comprise an amino acid sequence about 80% to about 100%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, or about 100%, identical to the amino acid sequence set forth in SEQ ID NO: 2.

The amino acid sequence of Cas6 may comprise SEQ ID NO: 3 or an equivalent thereof. The amino acid sequence of Cas6 may comprise an amino acid sequence about 80% to about 100%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, or about 100%, identical to the amino acid sequence set forth in SEQ ID NO: 3.

In an aspect the transposase is a Tn7 transposon comprising i) Transposon 7 protein A (TnsA) TnsA, ii) Transposon 7 protein B (Tns B), iii) Transposon 7 protein C (Tns C), and iv) transposition of integron protein Q (TniQ), and wherein the transposase is derived from Vibrio cholerae Tn6677. In an aspect, each of said right and left transposon end sequences comprises at least one TnsB binding site.

The term “transposon” encompasses a DNA segment with cis-acting sites (which may contain heterologous DNA sequences), and the genes that encode trans-acting proteins that act on those cis-acting sites to mobilize the DNA segment defined by the sites, regardless of how they are organized in DNA.

As used herein, the term “Tn7 transposon” refers to the prokaryotic transposable element Tn7, and modified forms or transposons sharing homology with Tn7 transposons (“Tn7-like transposons”). Tn7 has been most commonly studied in Escherichia coli. “Tn7 transposon” can encompass forms of DNA that do not demonstrably contain Tn7 genes, but which can be made to undergo transposition through use of the Tn7 gene products TnsA and TnsB, which collaborate to form the Tn7 transposase, or modifications thereof.

The Tn7 transposon contains characteristic left and right transposon end sequences and encodes five tns genes, tnsA-E, which collectively encode a heteromeric transposase, TnsA and TnsB which are catalytic enzymes that excise the transposon donor via coordinated double-strand breaks; TnsB, a member of the retroviral integrase superfamily, catalyzes DNA integration; TnsD and TnsE constitute mutually exclusive targeting factors that specify DNA integration sites; and TnsC is an ATPase that communicates between TnsAB and TnsD or TnsE. TnsD mediates site-specific Tn7 transposition into a conserved Tn7 attachment site (attTn7) downstream of the glmS gene in E. coli, whereas TnsE mediates random transposition into the lagging-strand template during replication. In E. coli, site-specific transposition involves attTn7 binding by TnsD, followed by interactions with the TnsC regulator protein to directly recruit the TnsA-TnsB-donor DNA. TnsC, TnsD, and TnsE interact with the target DNA to modulate the activity of the transposase via two distinct pathways. TnsABC+TnsD directs transposition to attTn7, a discrete site on the E. coli chromosome, at a high frequency, and to other loosely related “pseudo att” sites at low frequency. The alternative combination TnsABC+E directs transposition to many unrelated non-attTn7 sites in the chromosome at low frequency and preferentially to conjugating plasmids. Thus, attTn7 and conjugable plasmids contain positive signals that recruit the transposon to these target DNAs. The alternative target site selection mechanisms enable Tn7 to inspect a variety of potential target sites in the cell and select those most likely to ensure its survival.

As used herein, the term “transposase” refers to an enzyme that catalyzes transposition.

As used herein, the term “transposition” refers to a complex genetic rearrangement process, involving the movement of a DNA sequence from one location and insertion into another, for example between a genome and a DNA construct such as a plasmid, a bacmid, a cosmid, and a viral vector.

U.S. Ser. No. 10/947,534 is incorporated by reference herein for its disclosure of Tns polynucleotides and polypeptides.

The amino acid sequence of TnsA may comprise the amino acid sequence set forth in SEQ ID NO: 4 or an equivalent thereof. The amino acid sequence of TnsA may comprise an amino acid sequence at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, or about 100%, identical to the amino acid sequence set forth in SEQ ID NO: 4.

The amino acid sequence of TnsB may comprise SEQ ID NO: 5 or an equivalent thereof. The amino acid sequence of TnsB may comprise an amino acid sequence at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, or about 100%, identical to the amino acid sequence set forth in SEQ ID NO: 5.

The amino acid sequence of TnsC may comprise SEQ ID NO: 6 or an equivalent thereof. The amino acid sequence of TnsC may comprise an amino acid sequence about 80% to about 100%, at least or about 70%, at least or about 75%, at least or about 80%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, or about 100%, identical to the amino acid sequence set forth in SEQ ID NO: 6.

In an aspect, each of said right and left transposon end sequences comprises at least one TnsB binding site.

In an aspect, the transposase is a Mu transposase, such as MuA, MuB, MuC, or a combination thereof.

The transposons may be one of the Mu family, e.g., transposon of bacteriophage Mu, a bacterial class III transposon of Escherichia coli. In some cases, this transposon exhibits high transposition frequency. The Mu bacteriophage with its approximately 37 kb genome is relatively large compared to other transposons. The Mu transposon may have left end and right end transposase (e.g., MuA) recognition sequences (designated “L” and “R”, respectively) that flank the Mu transposable cassette, the region of the transposon that is ultimately integrated into the target site. In some examples, these ends are not inverted repeat sequences. The Mu transposable cassette, when necessary, may include a transpositional enhancer sequence (also referred to herein as the internal activating sequence, or “IAS”) located approximately 950 base pairs inward from the left end recognition sequence.

In some examples, a Mu transposon may have a 22 bp symmetrical consensus sequence, located near both ends, for recognition by a Mu transposase (MuA). Random transposition of a Mu transposon into a target gene occur through (1) binding of transposase (e.g., MuA) monomers to the Mu transposon recognition sites to form transposome assemblies, (2) tetramerization of the bound transposase (e.g., MuA) monomers to bridge the ends of the Mu transposon and engage the Mu transposon cleavage sites, (3) subsequent self-cleavage of the Mu transposon at the cleavage sites, and (4) accurate occurrence of a 5 bp staggered cut in a host DNA sequence into which the Mu transposon is subsequently incorporated.

The transposases may be Mu transposase family. Examples of transposases in the Mu family include MuA, MuB, and MuC.

In some examples, MuA may be a about 75-kDa multidomain protein (about 663 amino acids) and can be divided into structurally and functionally defined major domains (I, II, III) and subdomains (Ia, Ib, Ig; IIa, IIb; Ilia, PIb). The N-terminal subdomain la promotes transpososome assembly via an initial binding to a specific transpositional enhancer sequence. The specific DNA binding to transposon ends, crucial for the transpososome assembly, is mediated through amino acid residues located in subdomains Ib and Ig. Subdomain Ila contains the critical DDE-motif of acidic residues (D269, D336 and E392), which is involved in the metal ion coordination during the catalysis. Subdomains IIb and Ilia participate in nonspecific DNA binding, and they appear important during structural transitions. Subdomain Ilia also displays a cryptic endonuclease activity, which is required for the removal of the attached host DNA following the integration of infecting Mu. The C-terminal subdomain IIIb is responsible for the interaction with the phage-encoded MuB protein, important in targeting transposition into distal target sites. This subdomain is also important in interacting with the host-encoded ClpX protein, a factor which remodels the transpososome for disassemble.

In some examples, MuA may catalyze the steps of transposition: (i) initial cleavages at the transposon-host boundaries (donor cleavage) and (ii) covalent integration of the transposon into the target DNA (strand transfer). These steps may proceed via sequential structural transitions within a nucleoprotein complex, a transpososome, the core of which contains four MuA molecules and two synapsed transposon ends. In vivo, the critical MuA-catalyzed reaction steps may also involve the phage-encoded MuB targeting protein, host-encoded DNA architectural proteins (HU and IHF), certain DNA cofactors (MuA binding sites and transpositional enhancer sequence), as well as stringent DNA topology. The reaction steps mimicking Mu transposition into external target DNA can be reconstituted in vitro using MuA transposase, 50 bp Mu R-end DNA segments, and target DNA as the only macromolecular components.

In some examples, MuA and variants include those disclosed by EBI accession No. UNIPROT:Q58ZD8 (SEQ ID NO: 7) which has 36% identity to wild type MuA protein. In some examples, MuB may be an ATP-dependent DNA binding protein, which is required for efficient transposition in vivo. Bacteriophage Mu transposition may be influenced by the ATP-utilizing protein MuB. In vitro, the MuA transposase may direct insertions into targets that are bound by MuB. In some cases, there is no particular sequence specificity to MuB binding. However, its distribution on DNA may not be random: MuB binding to target molecules that already contain Mu sequences is specifically destabilized through an ATP-dependent mechanism. In some examples, MuB also stimulates the DNA-breakage and DNA-joining activities of MuA.

In some examples, the system comprises MuA. In some examples, the system comprises MuB. In some examples, the system comprises MuC. In some examples, the system comprises MuA and MuB. In some examples, the system comprises MuA and MuC. In some examples, the system comprises MuB and MuC. In some examples, the system comprises MuA, MuB, and MuC. In some examples, the system comprises a polynucleotide encoding MuA. In some examples, the system comprises a polynucleotide encoding MuB. In some examples, the system comprises a polynucleotide encoding MuC. In some examples, the system comprises a polynucleotide encoding MuA and a polynucleotide encoding MuC. In some examples, the system comprises a polynucleotide encoding MuA and a polynucleotide encoding MuC. In some examples, the system comprises a polynucleotide encoding MuB and a polynucleotide encoding MuC. In some examples, the system comprises a polynucleotide encoding MuA, a polynucleotide encoding MuB, and a polynucleotide encoding MuC.

WO2021/041922 is incorporated by reference herewith for its disclosure of Mu transposases and sequences of Mu polynucleotides and polypeptides.

The second non-replicating donor plasmid comprises an antibiotic selection marker, a guide RNA (gRNA) sequence and a cargo sequence flanked by a right transposon end sequence and a left transposon end sequence, wherein the gRNA is specific for a target site, and wherein the cargo sequence comprises a promoter, a protein expression sequence, or a combination thereof.

Exemplary antibiotic selection markers include genes encoding selection for ampicillin, chloramphenicol, kanamycin, gentamicin, apramycin, hygromycin, streptomycin, and the like.

Exemplary cargo sequences include promoters, protein expression sequences, and the like.

In an aspect, the promoter is one described in US2022-0119810, incorporated herein by reference for its disclosure of synthetic promoters and methods of selecting promoters.

The gRNA may be a crRNA/tracrRNA (or single guide RNA, sgRNA). The terms “gRNA,” “guide RNA” and “CRISPR guide sequence” may be used interchangeably throughout and refer to a nucleic acid comprising a sequence that determines the binding specificity of the CRISPR-Cas system. A gRNA hybridizes to (complementary to, partially or completely) a target nucleic acid sequence (e.g., the genome) in a host cell. The gRNA or portion thereof that hybridizes to the target nucleic acid (a target site) may be between 15-25 nucleotides, 18-22 nucleotides, or 19-21 nucleotides in length. In some embodiments, the gRNA sequence that hybridizes to the target nucleic acid is 15, 16, 17, 18, 19, 20, 21, 22, 23, 24, or 25 nucleotides in length. In some embodiments, the gRNA sequence that hybridizes to the target nucleic acid is between 10-30, or between 15-25, nucleotides in length. gRNAs or sgRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer). In one embodiment, gRNAs or sgRNA(s) can be between about 15 and about 30 nucleotides in length (e.g., about 15-29, 15-26, 15-25; 16-30, 16-29, 16-26, 16-25; or about 18-30, 18-29, 18-26, or 18-25 nucleotides in length).

To facilitate gRNA design, many computational tools have been developed.

In addition to a sequence that binds to a target nucleic acid, in some embodiments, the gRNA may also comprise a scaffold sequence (e.g., tracrRNA). In some embodiments, such a chimeric gRNA may be referred to as a single guide RNA (sgRNA).

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence.

In some embodiments, the gRNA sequence is at least 50%, 55%, 60%, 65%, 70%, 75%, 80%, 85%, 90%, 95%, 96%, 97%, 98%, 99%, or at least 100% complementary to a target nucleic acid. In some embodiments, the gRNA sequence is at least 50%, 55%, 60%, 65%, 70%, 75%, 80%, 85%, 90%, 95%, 96%, 97%, 98%, 99%, or at least 100% complementary to the 3′ end of the target nucleic acid (e.g., the last 5, 6, 7, 8, 9, or 10 nucleotides of the 3′ end of the target nucleic acid).

The gRNA may be a non-naturally occurring gRNA.

The target nucleic acid may be flanked by a protospacer adjacent motif (PAM). A PAM site is a nucleotide sequence in proximity to a target sequence. For example, the PAM may be a DNA sequence immediately following the DNA sequence targeted by the CRISPR/Cas system.

The target sequence may or may not be flanked by a protospacer adjacent motif (PAM) sequence. In certain embodiments, a nucleic acid-guided nuclease can only cleave a target sequence if an appropriate PAM is present. A PAM can be 5′ or 3′ of a target sequence. A PAM can be upstream or downstream of a target sequence. In one embodiment, the target sequence is immediately flanked on the 3′ end by a PAM sequence. A PAM can be 1, 2, 3, 4, 5, 6, 7, 8, 9, 10 or more nucleotides in length. In certain embodiments, a PAM is between 2-6 nucleotides in length. The target sequence may or may not be located adjacent to a PAM sequence (e.g., PAM sequence located immediately 3′ of the target sequence) (e.g., for Type I CRISPR/Cas systems and Type II CRISPR/Cas systems). In some embodiments, e.g., Type I systems, the PAM is on the alternate side of the protospacer (the 5′ end).

Non-limiting examples of the PAM sequences include: CC, CA, AG, GT, TA, AC, CA, GC, CG, GG, CT, TG, GA, AGG, TGG, T-rich PAMs (such as TTT, TTG, TTC, TTTT (SEQ ID NO: 8), etc.), NGG, NGA, NAG, NGGNG (SEQ ID NO: 9) and NNAGAAW (W=A or T, SEQ ID NO: 10), NNNNGATT (SEQ ID NO: 11), NAAR (R=A or G) (SEQ ID NO: 12), NNGRR (R=A or G) (SEQ ID NO: 13), NNAGAA (SEQ ID NO: 14) and NAAAAC (SEQ ID NO: 15), where “N” is any nucleotide.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization. There may be mismatches distal from the PAM.

In an aspect, the gRNA binds the transcribed strand of the target site. As shown herein, higher efficiencies can be achieved when the gRNA binds the transcribed strand of the target site.

In an aspect, the bacteria comprises Zymomonas mobilis, Klebsiella pneumoniae, Acinetobacter baumannii, Pseudomonas aeruginosa, Enterobacter cloacae, Pseudomonas putida, Shewanella oneidensis, E. coli, and the like.

As shown herein, a pool of second non-replicating donor plasmids can be employed wherein each member of the pool can include a different gRNA. In this way, both the transcribed and untranscribed strands can be targeted in a single experiment, and/or a plurality of target sites in the bacterial cell's genome can be targeted in a single experiment. Further, a plurality of cargoes such as promoters of varying strengths can be tested in a single experiment.

Thus, in an aspect, the system can comprise a pool of second non-replicating donor plasmids, wherein each member of the pool comprises the antibiotic selection marker, a member guide RNA (gRNA) sequence and a member cargo sequence flanked by the right transposon end sequence and the left transposon end sequence, wherein each member gRNA is specific for a target site, and wherein each member cargo sequence comprises a promoter, a protein expression sequence, or a combination thereof.

In this aspect, the member guide RNA sequences are specific for a single target site, or a plurality of target sites.

Also in this aspect, the member cargo sequences can be a plurality of promoters, particularly a plurality of constitutive promoter sequences of different strengths.

In this way, a gradient of overexpression of target genes can be achieved. This method is particularly useful for identifying target genes of both characterized and uncharacterized antibiotics.

In an aspect, the targeted gene is a protein coding gene involved in the mechanism of action of an antibiotic or provides resistance to growth inhibition during biofuel production.

In another aspect, the pooled second non-replicating donor plasmids can be used to screen all or substantially all protein coding genes in a bacterial genome. In this way, antibiotic-relevant gene phenotypes can be identified at the genome scale. For example, genes providing antibiotic resistance or susceptibility can be identified. Further, genes encoding antibiotic targets can be identified from pooled CRISPRtOE screens enabling antibiotic Mode of Action studies.

In another aspect, the pooled second non-replicating donor plasmids can be used to optimize bacterial strains for industrial use, such as identifying strains that resist growth inhibitors that can be present during biofuel production or identifying previously unidentified genes that influence biofuel production. For example, genes that provide resistance to toxins found in lignocellulosic feedstocks can be identified in pooled CRISPRtOE screens that increase growth in such feedstocks. The identity of the growth inhibitor(s) in the feedstock does not need to be known to identify strains that bypass inhibitor toxicity.

The invention is further illustrated by the following non-limiting examples.

### EXAMPLES

### Methods

Strains and growth conditions. Growth and antibiotic selection conditions are also summarized in Table 1. Escherichia coli, Acinetobacter baumannii, Enterobacter cloacae, Klebsiella pneumoniae, and Pseudomonas aeruginosa were grown in LB broth, Lennox (BD240230) at 37° C. (or 30° C. for CRISPR-guided transposition) in a flask with shaking at 250 rpm, in a culture tube on a roller drum, in a 96 well deepwell plate with shaking at 900 rpm, or in a Tecan Sunrise plate reader shaking with OD600 measurements every 15 min. Pseudomonas putida and Shewanella oneidensis were grown in LB in a culture tube on a roller drum or in a 96 well deepwell plate with shaking at 900 rpm at 30° C. Zymomonas mobilis was grown in RMG medium (10 g yeast extract and 2 g KH₂PO₄ monobasic/liter with 2% glucose added after autoclaving) at 30° C. statically in a culture tube or deepwell plate. E. coli was grown in Mueller Hinton Broth (MHB, BD 275730) for antibiotic minimal inhibitory concentration (MIC) assays. Media was solidified with 1.5-2% agar for growth on plates. Antibiotics were added when necessary: E. coli (100 μg/ml carbenicillin (carb), 30 μg/ml kanamycin (kan), or 50 μg/ml spectinomycin (spt)), A. baumannii (60 μg/ml kan or 100 μg/ml apramycin (apr)), E. cloacae (30 μg/ml kan or 50 μg/ml spt), K. pneumoniae (60 μg/ml kan or 50 μg/ml spt), P. aeruginosa (30 μg/ml gentamicin (gen) or 1 mg/ml kan), P. putida (150 μg/ml apr or 60 μg/ml kan), S. oneidensis (60 μg/ml kan or 30 μg/ml gen), and Z. mobilis (200 μg/ml gen or 100 μg/ml spt). Diaminopimelic acid (DAP) was added at 300 μM to support growth of dap-E. coli strains. Strains were preserved in 15% glycerol at −80° C.

TABLE 1
Growth and antibiotic selection conditions

| | | | | Antibiotic for | Antibiotic |
|---|---|---|---|---|---|
| | | Taxonomy/ | Growth | CRISPRt/ | for Tn7 |
| Organism | Strain | category | conditions | CRISPRtOE* | reporter* |
| Acinetobacter | 17978 | Gammaproteo | LB, 37° C. | kan(60) | apr(100) |
| baumannii | | bacterium, | | | |
| | | ESKAPE | | | |
| | | pathogen | | | |
| Enterobacter | CDC 442-68 | Gammaproteo | LB, 37° C. | kan(30) | spt(50) |
| cloacae | | bacterium, | | | |
| | | ESKAPE | | | |
| | | pathogen | | | |
| Escherichia | MG1655 | Gammaproteo | LB, 37° C. | kan(30) | spt(50) |
| coli | | bacterium, | | | |
| | | model | | | |
| Klebsiella | KPPR1 | Gammaproteo | LB, 37° C. | kan(60) | spt(50) |
| pneumoniae | | bacterium, | | | |
| | | ESKAPE | | | |
| | | pathogen | | | |
| Pseudomonas | PA14 | Gammaproteo | LB, 37° C. | gm(30) | kan(1000) |
| aeruginosa | | bacterium, | | | |
| | | ESKAPE | | | |
| | | pathogen | | | |
| Pseudomonas | KT2440 | Gammaproteo | LB, 30° C. | apr(150) | kan(60) |
| putida | | bacterium, | | | |
| | | industrial | | | |
| Shewanella | MR-1 | Gammaproteo | LB, 30° C. | kan(60) | gm(30) |
| oneidensis | | bacterium, | | | |
| | | industrial | | | |
| Zymomonas | ZM4 | Alphaproteo | RMG, 30° C. | gm(200) | spt(100) |
| mobilis | | bacterium, | | | |
| | | industrial | | | |

*antibiotic concentrations in μg/ml, apramycin (apr), gentamicin (gen), kanamycin (kan), spectinomycin (spt)

General molecular biology techniques. pir-dependent plasmids were propagated in E. coli strain BW25141 (sJMP146) or its derivative sJMP3053. Plasmids were purified using the GeneJet™ Plasmid Miniprep kit (Thermo K0503), the QIAprep® Spin Miniprep Kit (Qiagen 27106), or the Purelink™ HiPure Plasmid Midiprep kit (Invitrogen K210005). Plasmids were digested with restriction enzymes from New England Biolabs (NEB, Ipswich, MA). Ligations used T4 DNA ligase (NEB M0202) and fragment assembly used NEBuilder® Hifi (NEB E2621). Genomic DNA was extracted with the DNeasy® Blood & Tissue Kit (Qiagen 69504) or the GeneJet™ genomic DNA purification kit (Thermo K0721). DNA fragments were amplified by PCR using Q5 DNA polymerase (NEB M0491, for cloning) or OneTaq® DNA Polymerase (NEB M0480, for analysis). PCR products were spin-purified using the Monarch® PCR & DNA Cleanup Kit (NEB T1030) or the DNA Clean & Concentrator®-5 kit (Zymo Research, Irvine, CA, D4013). Reactions were purified with 1.8× Mag-Bind® TotalPure NGS magnetic beads (Omega) on a magnetic rack (Alpaqua). DNA was quantified spectrophometrically using a NanoDrop™ Lite or fluorometrically using a Qubit™ with the HS or BR DNA kit (Thermo). Plasmids were transformed into electrocompetent E. coli cells using a 0.1 cm cuvette (Fisher FB101) and a BioRad Gene Pulser Xcell™ on the Bacterial 1 E. coli preset protocol (25 F, 200 ohm, 1800 V) as described in detail in the art. Oligonucleotides were synthesized by Integrated DNA Technologies (Coralville, IA) or Agilent (SurePrint Oligonucleotide library) (Santa Clara, CA). Sequencing was performed by Functional Biosciences (Madison, WI), Plasmidsaurus (Eugene, OR), Azenta (South Plainfield, NJ), or the replaced by an mScarlet-I encoding gene derived from Addgene plasmid 85069 (pJMP7001) to create pJMP10180 which has an mScarlet-I cassette (BbsI sites removed and mScarlet converted to mScarlet-1) with the T7A1 constitutive promoter in the Tn7 transposon. A promoterless mScarlet-I reporter with the CRISPRt LZ1 protospacer was derived from pJMP6957, pJMP10180, and synthetic DNA oJMP1599 to create pJMP10206.

Construction of CRISPRt/CRISPRtOE system. The CRISPRt/CRISPRtOE donor plasmids were derived from pSpin® (pSL1765), Mobile-CRISPRi plasmids, and synthetic DNA. Cloning details not shown. In summary, (1) a functional antibiotic resistance cassette was added to the transposon (pJMP747) and the BsaI cloning site in the gRNA cassette was altered (pJMP10009 and pJMP10011). A two plasmid R6K ori (pir-dependent) system for selectable CRISPR-guided transposition was created by combining (1) the TnsABC-TniQ-Cas876 expression cassette from pSpin® with the amp^{R}, R6K ori, mobilizable backbone from Mobile-CRISPRi (pJMP2782) to create pCRISPRt-H (pJMP10233) and (2) the transposon and guide RNA cassette from pJMP10011 with the same backbone (from pJMP2782) to create pCt-T (pJMP10237 and derivatives) which was further minimized, optimized, and altered by adding transcription terminators and moving the guide RNA cassette inside the transposon to produce pCRISPRt-T-gent (pJMP10395) and pCRISPRt-T-kan (pJMP10397). CRISPRtOE plasmids with either no promoter or various strength promoters (pCtOE-noP, pCtOE-PA, pCtOE-PH, pCtOE-Pv) were derived from these plasmids by inserting a transcription terminator and promoter cassette derived from synthetic DNA (oJMP2000 plus oJMP2001-2004) into the above plasmids to produce plasmids oJMP10471-10479. Apramycin resistant derivatives of some of these plasmids were created by digesting pJMP10397 and pJMP10478 with XhoI and assembling with a piece of synthetic DNA (oJMP1946) encoding an apramycin resistance gene.

CRISPRtOE Guide Design. Guide RNAs were designed using custom Python scripts, available with usage notes at GitHub repositories. Initially, the Escherichia coli K-12 RefSeq genome assembly, in GenBank file format, was downloaded from the NCBI database (Accession Number: GCF_000005845.2), using the seq_hunter.py script. The CRISPRt_gRNA.py script was employed to identify all genomic locations with a suitable “CN” PAM sequence. Subsequently, potential spacers of 32 nucleotides in length were evaluated using awk, and ten were selected based on the following criteria: 1) Unique occurrence in the genome, 2) occurrence on the same strand as the downstream gene, 3) at least 95 nucleotides upstream from the gene annotation.

CRISPRt/CRISPRtOE individual guide and guide library construction. Guide sequences were ligated into the BsaI-cloning site of the pCRISPRt-T/pCtOE plasmids. For cloning individual spacer sequences, two 36-nucleotide (nt) oligonucleotides (oligos) were annealed so that they encode the desired sequence flanked by sticky ends compatible to the vector BsaI site whereas for cloning pooled libraries, DNA fragments encoding spacers were amplified from a pool of oligos and digested with BsaI prior to ligation.

To prepare the pCRISPRt-T/pCtOE vectors for cloning, plasmid DNA was extracted from a 100 ml culture using a midiprep kit, 2-10 μg plasmid DNA was digested with BsaI-HF-v2 (NEB R3733) in a 100 μl reaction for 4 h at 37° C. and then spin-purified.

For cloning individual guides, pairs of oligonucleotides (2 μM each) were mixed in a 50 μl total volume of 1×NEB CutSmart® buffer, heated at 95° C., 5 min and then cooled to room temperature ˜20 min to anneal, and then diluted 1:40 in dH₂O prior to ligation. Ligation reactions (10 μl) contained 1×T4 DNA ligase buffer (NEB), additional DTT (10 mM final) and ATP (0.1 mM final), and 0.5 μl T4 DNA ligase in addition to 50 ng BsaI-digested/spin-purified vector and 2 μl of 1:40 diluted annealed oligos. Ligations were incubated at 25° C. for 2 h and ligase was heat inactivated for 20 min at 65° C. 1-2 μl ligation was used for electroporation into 50 μl E. coli strain BW25141 (sJMP146). Transformations were serially diluted and plated with selection on carbopenicillin to obtain isolated colonies. After confirmation of sequence, individual plasmids were transformed into E. coli mating strain WM6026 (sJMP424 or sJMP3257) with selection on carb and DAP.

For cloning pooled libraries, fragments were amplified from 90 nt pooled oligos (IDT oPools™ or Agilent SurePrint pools) using the following conditions per 100 μl reaction (reaction size was adjusted ˜100-600 μl depending on size of library): 20 μl Q5 buffer, 3 μl GC enhancer, 2 μl 10 mM each dNTPs, 5 μl each 10 μM forward and reverse primers, 2 μl 10 nM pooled oligo library, 1 μl Q5 DNA polymerase, and 186 μl H₂O with the following thermocycling parameters: 98° C., 30 s; 15-19 cycles of: 98° C., 15 s; 56° C., 15 s; 72° C., 15 s; 72° C., 10 min; 10° C., hold (cycles were adjusted to obtain sufficient PCR product without overamplifying/biasing the library). PCR products (90 bp) were spin-purified and 300 ng was digested with BsaI-HF-v2 (NEB R3733) in a 100 μl reaction for 2 h at 37° C. (no heat kill, reaction size was adjusted proportionally depending on the size of the library). Size and digestion of PCR products were confirmed on a 4% agarose E-Gel™ (Thermo). The BsaI-digested PCR product without further purification (3 ng/μl) was ligated into BsaI-digested, spin-purified plasmid as described above except 10 μl of the digested PCR product was ligated with 500 ng cut plasmid in a 100 μl reaction and ligations were incubated at 16° C. for 14 h. Library ligations were purified by spot dialysis on a nitrocellulose filter (Millipore VSWP02500) against 0.1 mM Tris, pH 8 buffer for 20 min prior to transformation by electroporation into E. coli mating strain WM6026 (sJMP424 or sJMP3257). To obtain sufficient transformants for large libraries, electrocompetent cells were made 5× more concentrated in the final step of the preparation and multiple transformations were carried out depending on the size of the library. Transformations were plated with selection on LB with carb and DAP at a density of ˜30,000-50,000 colonies/150 mm petri plate. Cells (>30× more colonies the number of guides, e.g. >1.35 million CFU for the 45,000 guide libraries) were scraped from plates and resuspended in LB+15% glycerol, density was adjusted to OD₆₀₀=9, and aliquots were frozen at −80° C.

Transfer of the CRISPRt/CRISPRtOE system to the bacterial chromosome. CRISPRt and CRISPRtOE strains were constructed by triparental mating of two E. coli donor strains-one with the pCRISPRt-H plasmid encoding Cas678-TniQ-TnsABC and another with the pCRISPRt-T or pCtOE plasmid containing the transposon with the guide RNA and antibiotic resistance- and a recipient strain (Table 1). All conjugations and selection post-conjugation were carried out at 30° C. regardless of the normal incubation temperature for the organism. Isolated donor and recipient strains were struck out on plates with the appropriate media and antibiotic (if relevant) concentrations. Colonies were resuspended from plates into the growth medium used for the recipient to a density of OD₆₀₀=9. Equal amounts of donors and recipient were mixed together (along with the appropriate no recipient, no CRISPRt-H donor, or no CRISPRt-T donor controls), cells were pelleted by centrifugation at 5000×g (except A. baumannii at 9000×g), placed on a nitrocellulose filter on an LB plate (except an RMG plate for Z. mobilis), and incubated 12-18 h at 30° C. Cells from the filter were resuspended in 300 μl media, serially diluted, and plated with antibiotic selection for the transposon (see growth and selection conditions above) and without DAP to select against the donor strains, and incubated at 30° C. until colonies formed (1-3 days). A typical mating was 100 μl each strain and plating 100 μl undiluted, and 10⁻¹ through 10⁻⁴ dilutions to obtain isolated colonies. For library construction, conjugations were scaled up and plated with selection at a density of ˜30,000-50,000 colonies/150 mm petri plate. Cells (>30× more colonies the number of guides, e.g. >1.35 million CFU for the 45,000 guide libraries) were scraped from plates and resuspended in LB+15% glycerol, density was adjusted to OD₆₀₀=10-15, and aliquots were frozen at −80° C.

Analysis of phenotype of E. coli lacZ and gfp-targeting CRISPRt. For gfp targeted CRISPRt, fluorescence level (GFP) of 80 individual colonies was determined. Cultures were grown in 300 μl media in 96 well deepwell plates from a single colony to saturation. Cells were centrifuged at 4,000×g for 10 min and cell pellets were resuspended in 300 μl 1×PBS. 150 μl was transferred to a 96-well black, clear bottom microplate (Corning 3631) and cell density (OD₆₀₀) and fluorescence (excitation 475 nm, emission 510 nm) were measured in a fluorescence microplate reader (Tecan Infinite® Mplex). Fluorescence values were normalized to cell density.

For lacZ targeted CRISPRt, the color of 80 individual colonies was determined by patching on LB agar plates containing X-gal (20 μg/ml). Blue indicates the presence of an intact lacZ due to metabolism of the X-gal by LacZ (betagalactosidase) and white indicates disruption of lacZ.

Analysis of individual CRISPRtOE isolates from diverse bacteria. Phenotype determination. Fluorescence levels (mScarlet-I) of 12 CRISPRtOE isolates of all 8 species were determined. Cultures of isolates were grown in 300 μl media in 96 well deepwell plates from a single colony to saturation. Cultures were serially diluted to 1:10,000 (1:100 twice) into fresh media and grown again to saturation (˜13 doublings). Cells were centrifuged at 4,000×g for 10 min. Cell pellets were resuspended in 300 μl 1×PBS and 150 μl was transferred to a 96-well black, clear bottom microplate (Corning 3631). C ell density (OD600) and fluorescence (excitation 584 nm, emission 607 nms) were measured in a fluorescence microplate reader (Tecan Infinite® Mplex). Fluorescence values were normalized to cell density. The assay was repeated three times for E. coli and Z. mobilis and twice for all other strains.

Genotype determination. Insertion positions of 12 CRISPRtOE isolates of all 8 species were determined. Cultures of isolates were grown in 300 μl media in 96 well deepwell plates, serially diluted 1:100 in dH₂O, heated to 90° C. for 3 min prior to use as a template for PCR. Fragments (˜2000 bp) were amplified by PCR with a forward primer in the transposon (oJMP490) and a set of indexed (4 nt barcode) reverse primers in the mScarlet-I coding sequence (oJMP2118-2129) in a 25 μl reaction containing: 5 μl OneTaq® buffer, 0.5 μl 10 mM each dNTPs, 0.5 μl each 10 μM forward and reverse primers, 2 μl diluted culture, 0.25 μl OneTaq® DNA polymerase, and 16.25 μl H₂O with the following touchdown PCR program: 94° C., 3 min then 10 cycles of 94° C., 30 s, 65° C., 30 s (−1° C./cycle), 68° C., 2.5 min, then 25 cycles of 94° C., 30 s, 55° C., 30 s, 68° C., 2.5 min, then 68° C., 5 min in a BioRad T100 thermalcycler. Sets of indexed PCRs for each organism were pooled, spin purified and sequenced by Oxford nanopore long read amplicon sequencing by Plasmidsaurus. Sequencing data was demultiplexed using a custom Python script (SplitSamplesSeq.py) and insertion position relative to the mScarlet-I ATG start codon was determined.

CRISPRtOE library growth experiments. Competition experiment growth. The E. coli CRISPRtOE murA and folA libraries were mixed in equal volume with the LP-lacZ library: 50 μl frozen stock (OD₆₀₀=10) of each library (murA+LP-lacZ and folA+LP-lacZ) into 100 ml LB (starting OD₆₀₀=0.01) in a 500 ml flask and incubated shaking at 37° C. until OD₆₀₀=0.2 (˜2.5 h) (timepoint=T0) to revive the cells. These cultures were diluted to OD₆₀₀=0.02 into 4 ml LB plus antibiotic fosfomycin (0.4 μg/ml, for the murA+LP-lacZ libraries) or trimethoprim (0.1 μg/ml, for the folA+LP-lacZ libraries) or no antibiotic control in 14 ml snap cap culture tubes (Corning 352059) in duplicate and incubated with shaking for 18 h at 37° C. (T1). These cultures were serially diluted back to OD₆₀₀=0.01 into fresh tubes containing the same media and incubated again with shaking for 18 h at 37° C. (T2) for a total of ˜10-15 doublings. Cells were pelleted from 15 ml of culture in duplicate at each time point T0 and 1 ml of culture at timepoints T1 and T2 and stored at −20° C.

Whole genome experiment growth. The E. coli CRISPRtOE whole genome libraries with no promoter (sJMP10704) or promoter H (sJMP10705) were revived by dilution of 100 μl frozen stock (OD₆₀₀=15) into 100 ml LB (starting OD₆₀₀=0.015) and incubation in 500 ml flasks shaking at 37° C. until OD₆₀₀=0.2 (˜2.5 h) (timepoint=TO). These cultures were diluted to OD₆₀₀=0.02 into 4 ml LB plus antibiotic trimethoprim (0.1 μg/ml) or no antibiotic control in 14 ml snap cap culture tubes (Corning 352059) in duplicate and incubated with shaking for 18 h at 37° C. (T1). These cultures were serially diluted back to OD₆₀₀=0.01 into fresh tubes containing the same media and incubated again with shaking for 18 h at 37° C. (T2) for a total of ˜10-15 doublings. Cells were pelleted from 15 ml of culture in duplicate at time point TO and 1 ml of culture at timepoints T1 and T2 and stored at −20° C.

Analysis of individual CRISPRtOE isolates from library competition experiment. Insertion positions of 8 CRISPRtOE isolates from the folA and murA libraries were determined. Cultures of isolates were grown in 3 ml media from an isolated colony, serially diluted 1:100 in dH₂O, heated to 90° C. for 3 min prior to use as a template for PCR. Fragments (˜600 bp) were amplified by PCR with a forward primer in the transposon (oJMP61) and a reverse primer in the folA or murA coding sequence (oJMP2349 and oJMP2348, respectively) in a 50 μl reaction containing: 10 μl OneTaq® buffer, 1.0 μl 10 mM each dNTPs, 1.0 μl each 10 μM forward and reverse primers, 2 μl diluted culture, 0.5 μl OneTaq® DNA polymerase, and 16.25 μl H₂O with the following touchdown PCR program: 94° C., 3 min then 10 cycles of 94° C., 30 s, 65° C., 30 s (−1° C./cycle), 68° C., 1 min, then 25 cycles of 94° C., 30 s, 55° C., 30 s, 68° C., 1 min, then 68° C., 5 min in a BioRad T100 thermal cycler. PCR products were spin-purified and Sanger sequenced to determine the transposon insertion position relative to the mScarlet-I ATG start codon.

Tnseq analysis of CRISPRt/CRISPRtOE constructs. TnSeq protocol was adapted from the art. Genomic DNA (gDNA) was extracted from the equivalent of 1 ml of cells at OD₆₀₀=3 (˜2×10⁹ cells) either resuspended from a plate or from liquid culture and was further purified by spot dialysis on a nitrocellulose filter (Millipore VSWP02500) against 0.1 mM Tris, pH 8 buffer for 20 min. 1 μg gDNA was digested with 4 U MmeI (NEB) in a 50 μl reaction at 37° C., 12 hrs and heat inactivated at 65° C., 20 min. The digest was purified using 1.8× magnetic beads (Omega) following the manufacturer's protocol, eluting in 20 μl 10 mM Tris, pH 8.0. Adapter oligos oJMP1995 and P-oJMP1996 (phosphorylated) were annealed by mixing equal volume of 100 μM oligos in 10 mM Tris, pH8 (50 μM each), heating to 95° C., 5 min followed by cooling to RT ˜15 min, and diluted 1:10 (5 μM each) for use. Annealed oligos were ligated onto MmeI-digested DNA in a 20 μl reaction with 2 μl 10× T4 ligase buffer, 2 μl 100 mM DTT, 2 μl 1 mM ATP, 12 μl purified MmeI-digested gDNA (50 ng/μl, 600 ng total), 1 μl 5 μM annealed adapter oligos (250 nM final) and 1 μl T4 DNA ligase. Ligations were incubated 14 h at 16° C. and the enzyme was heat-inactivated for 20 min at 65° C. Ligations were purified using 1.8× magnetic beads, eluting in 22 μl 10 mM Tris, pH 8. Fragments were amplified by PCR with primers containing partial adapters for index PCR with Illumina® TruSeq™ adapters in a 100 μl reaction containing: 20 μl 5×Q5 buffer, 3 μl GC enhancer, 2 μl 10 mM each dNTPs, 5 μl each forward primer (oJMP1997) and reverse primer (oJMP1998 or indexed primers oJMP2022-2033), 10 μl purified ligation, 1 μl Q5 DNA polymerase, and 54 μl dH₂O with the following program: 98° C., 30 s then 18-20 cycles of 98° C., 15 s, 66° C., 15 s, 72° C., 15 s, then 72° C., 10 min in a BioRad C1000 thermal cycler. PCR products were spin-purified (eluted in 15 μl) and quantified fluorometrically. Samples were sequenced by the UWBC NGS Core facility or Azenta Amplicon-EZ service. Briefly, PCR products were amplified with nested primers containing i5 and i7 indexes and Illumina® TruSeq™ adapters followed by bead cleanup, quantification, pooling and running on a NovaSeq X Plus (150 bp paired end reads) or MiSeq (250 bp paired end reads). Sequencing analysis of the initial CRISPRt or CRISPRtOE plasmid libraries from which the strain libraries were prepared was by amplification with oJMP2011 and oJMP1998 (or barcoded version of oJMP1998: oJMP2022-2033), followed by spin purification and Illumina® sequencing.

Analysis of E. coli mScarlet-I-targeting CRISPRt libraries. Two plasmid libraries (pJMP10505 and pJMP10506) were constructed (gen^{R} and kan^{R}) and used to create CRISPRtOE libraries (sJMP10519, 10520, 10594, 10595, and 10637-10640) in each of four E. coli mScarlet-I reporter strains (sJMP10205, 10269, 10630, 10633). Plasmid DNA from pJMP10505 and pJMP10506 was amplified with oJMP2011 and oJMP1998. gDNA from (sJMP10519, 10520, 10594, 10595, and 10637-10640) was processed by TnSeq as detailed above with oJMP1997 and oJMP2022-2033. Sequencing data was demultiplexed using SplitSamplesSeq.py and guides were quantified using seal.sh from bbmap.

MIC assays. The minimal inhibitory concentration (MIC) of E. coli folA and murA CRISPRtOE isolates was determined by either growth in a microtiter plate or by disc diffusion assays, respectively. For the broth microdilution assay, CRISPRtOE isolates and WT controls were grown to saturation from an isolated colony and serially diluted to OD600=0.003) and the CRISPRtOE library was diluted from a glycerol stock to the same density. Trimethoprim was serially diluted in DMSO at 1000× concentration and then diluted to 2× in MHB. E. coli folA CRISPRtOE isolates, the folA CRISPRtOE library, a lacZ CRISPRtOE control, and WT cultures and the media containing antibiotic were mixed in equal proportions and incubated 20 h shaking at 37° C. prior to cell density (OD₆₀₀) determination in a microplate reader. For the MIC strip assays, E. coli folA or murA CRISPRtOE isolates, a lacZ CRISPRtOE control, and WT cultures were diluted to OD₆₀₀=0.3 and 300 μl was spread on a 100 mm MH agar plate and dried in a laminar flow cabinet. When dry, MIC strips (Liofilchem, Italy; 920371 (TMP 0.002-32 mg/L) and 920791(FOS 0.064-1024 mg/L)) were applied to the plate and 10 μl serially diluted antibiotics were applied to the discs. Plates were incubated at 37° C. for 18 h. Broth microdilution assays were repeated 3 times and MIC test strip assays twice.

Cutadapt (v3.4) using default parameters and sequences flanking the desired genome sequence. The trimmed FASTQ files were aligned to the E. coli genome (NCBI RefSeq Assembly ID GCF_000005845.2) using Bowtie2 (v2.4.4)⁶¹ using default parameters for paired-end alignment. The alignment SAM file for each sample was filtered using Samtools (v1.13) to remove unmapped reads (-F 8) and to remove reads that mapped to opposite strands (-F 0x2). This resulted in paired end reads that aligned to the same DNA strand. BBMap (v38.32) was used to filter the SAM files to remove read pairs wherein at least one read is less than 2 nucleotides or greater than 50 nucleotides in order to identify specific transposon insertion sites downstream of spacer sequences. A custom Python script (parsing_sam_for_hisogram.py) and a custom Rscript (Histogram_CRISPRtOE.R) were used to parse the filtered SAM files and construct histograms representing the distance between the spacer sequence and the transposon insertion site.

The SAM files for each sample were further filtered to remove reads there were more than 100 nucleotides apart using the Picard tools (v2.20) “FilterSamReads” function with default parameters. This resulted in an aligned file where in the spacer sequence was located on the same strand as the transposon insertion site and in the same location (within 100 nucleotides). These filtered files were used for all further analyses. Samtools (v1.13)⁶² was used to sort and index these files and DeepTools (v3.5.1) was used to construct BigWig files (normalized using CPM) and Bed files for visualization. To identify the distance between the transposon insertion site and the nearest gene downstream, the alignment file was filtered to include only reads <25 nucleotides (those reads corresponding to the site of transposon insertion) using Samtools (v1.13) and a custom Python script (site_of_insertion.py) was used to generate a resulting table of the location of transposon insertion sites. The table was converted to a Bed file using a custom Python script (table_to_bed.py) and the BEDOPS (v2.4.41) command “closest-features” was used to identify the distance between transposon insertion site and the nearest gene downstream. CRISPRtOE distance results from all samples were combined and a custom Python script (make_table_for_histogram.py) was used to construct the input for R. To compare CRISPRtOE insertion sites relative to genes to that of traditional Tn-seq, Tn-seq data from Goodall et al. (TL1 samples) were aligned to the same E. coli genome (NCBI RefSeq Assembly ID GCF_000005845.2) using Bowtie2 (v2.4.4) using default parameters and Bed files were created using DeepTools (v3.5.1) as described above. The Tn-seq data was processed to identify the distance between transposon insertion and the gene downstream and compared to CRISPRtOE distance as above. Data were visualized on the IGV Genome Browser and Circos plots were created using Proksee.

A custom Python script (counting_spacers.py) was used to determine the number of times each spacer appeared in the filtered alignment file. Spacers were retained for analysis if they had a count ≥5. Pairwise comparison of spacer counts in DMSO and TMP treatment samples was performed using edgeR⁷⁰ and a custom Rscript (comparison_of_spacer_counts_edgeR.R). The results were plotted using a custom Rscripts (plotting_scatter_plot.R, barplot_with_points.R, scatter_plot_commands.R). For gene analysis, spacers for each gene were averaged and genes were retained if they contained ≥2 spacers for further analysis.

CRISPRtOE data processing and analysis. All commands and scripts used for processing and analysis of CRISPRtOE data can be found on GitHub (https://github.com/GLBRC/CRISPRtOE_Analysis). FASTQ files were separated for each read (R1 and R2), concatenated, and trimmed to remove transposon-specific sequences using

### Example 1: A Vc CAST System Optimized for Functional Genomics

“CRISPRt,” a Vc CAST system optimized for functional genomics, was generated. Like previous Vc CAST systems, CRISPRt enables precise insertion of transposons into genomic targets using programmable gRNAs (FIG. 1b). Superimposed on this are additional properties necessary for performing functional genomics: 1) The Tn insertion is selectable; 2) cas/Tn plasmid replication in recipient strains is not required for transposition; 3) following transposition, no exogenous cas genes or plasmid components outside of the Tn remain in the recipient; and 4) Tn cargo is facile to modify due to separation of transposase/cas and transposon elements across two plasmids (FIG. 1a, 1b, 1c). CRISPRt can be transferred from donor Escherichia coli K-12 cells to diverse recipient bacteria via conjugation, and transconjugants containing Tn insertions are recovered at efficiencies consistent with genome-scale library creation.

To test the precision of CRISPRt, both endogenous (lacZ) and heterologous (gfp) reporter genes were targeted for disruption in E. coli K-12 (data not shown). Consistent with previous data on Vc CAST systems, the vast majority of Tn insertions were on-target. First, targeting of gfp and lacZ were assessed using phenotypic assays (fluorescence measurements for gfp and blue-white screening for lacZ), finding that 97.6% and 100% of transconjugants showed the expected disruption phenotype, respectively (data not shown). Next, targeting on the genome scale was determined by sequencing genomic DNA at the Tn6677 transposon junction (Tn-seq) using Illumina®-based Next Generation Sequencing (NGS). Tn-seq showed >99.5% on target efficacy for both gfp and lacZ guide RNAs (FIG. 1d). As expected, Tn insertions largely occurred in a tight window centered at ˜49 bp downstream of the protospacer that favored an R-L orientation (89.2% and 74.1% R-L for gfp and lacZ, respectively, (FIGS. 1d and 1e)). Moreover, CRISPRt transconjugants were recovered at an efficiency compatible with genome-scale screening (˜10⁻² to 10⁻³ in E. coli, (data not shown)). CRISPRt has excellent properties for functional genomics while retaining the on-target efficacy of Vc CAST.

### Example 2: Targeting Non-Transcribed DNA Improves CRISPRt Efficacy

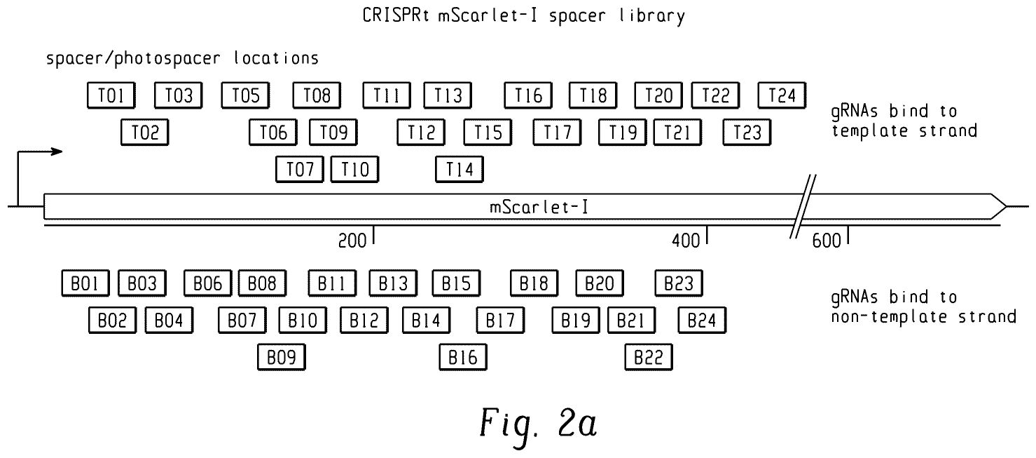

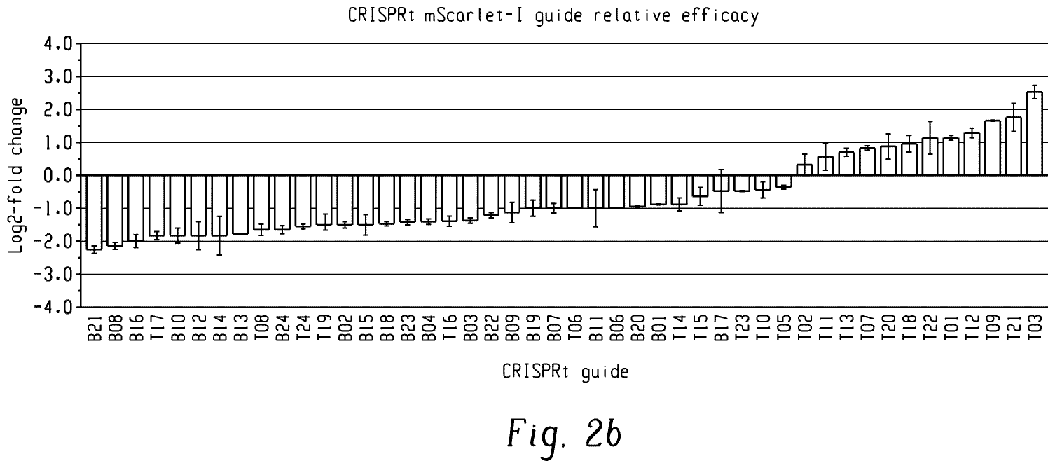

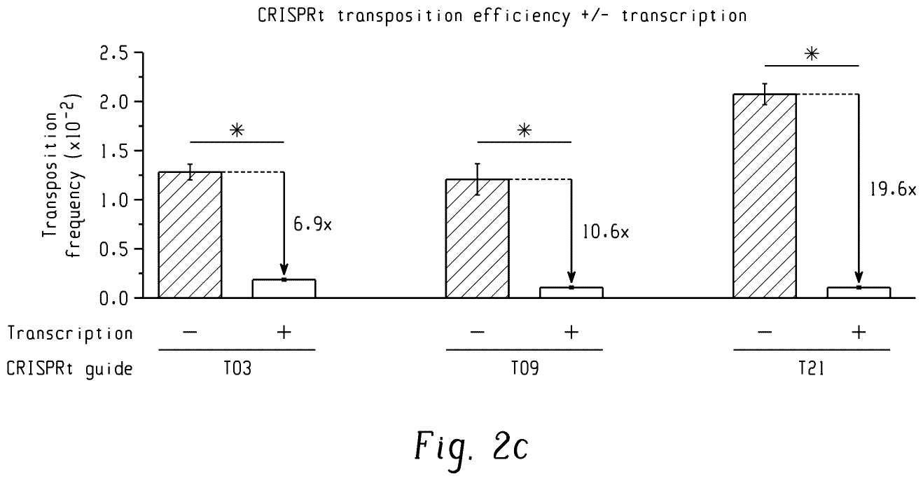

Although Protospacer Adjacent Motif (PAM) requirements for Vc CAST have been well characterized (a “CN” PAM is sufficient), other genomic features that impact guide efficacy are poorly understood. To address this issue, a pooled CRISPRt library targeting both strands of an mScarlet-I reporter gene that was integrated into the genome of E. coli K-12 was created (46 gRNAs total (FIG. 2a)). Surprisingly, guide efficacy depended on which strand was targeted. To measure relative guide efficacy, the abundance of gRNA spacers in the library before and after transposition into mScarlet-I was determined (i.e., in mating donor strains versus recipient transconjugants (FIG. 2b)). Consistent with previous data from smaller sets of spacers, considerable variability was observed, with ˜27-fold difference in efficacy between the most and least active guides. A clear, but not absolute bias toward higher efficacy in template strand targeting guides (i.e., gRNAs that bind the transcribed strand) versus those that targeted the non-template strand was observed. Other variables, such as PAM identity or distance from the gene start did not show an obvious trend (data not shown).

The bias in strand specificity suggested that either replication or transcription could be impacting CRISPRt efficacy. To test for a replication effect, the orientation of mScarlet-I relative to the origin or replication was flipped (data not shown). However, strand specificity was observed in the flipped target, seemingly ruling out a role for replication direction in CRISPRt efficacy (data not shown). To test for a transcription effect, a promoterless version of mScarlet-I was targeted with the CRISPRt library (data not shown). In contrast to the transcribed target, the promoterless mScarlet-I gene showed reduced variation in guide efficacy, consistent with transcription affecting CRISPRt function ( ). Because the pooled approach did not allow for an absolute measure of CRISPRt activity, the three most active guides from the mScarlet-I library (T03, T09, and T21) were cloned their activities individually tested (FIG. 2c). Transcription reduced recovery of CRISPRt transconjugants (transposition efficiency) by 6.9 to 19.6-fold, depending on the guide tested. Although Tn disruption of transcribed genes is effective, CRISPRt efficacy increases on non-transcribed targets. This finding indicates that CRISPRt is ideal for targeting intergenic DNA.

### Example 3: Targeted and Tunable Overexpression in Diverse Bacteria

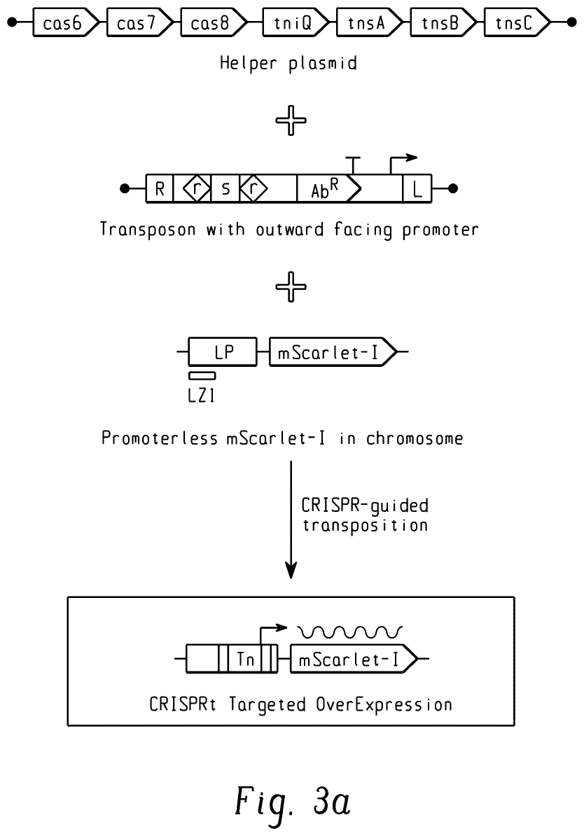

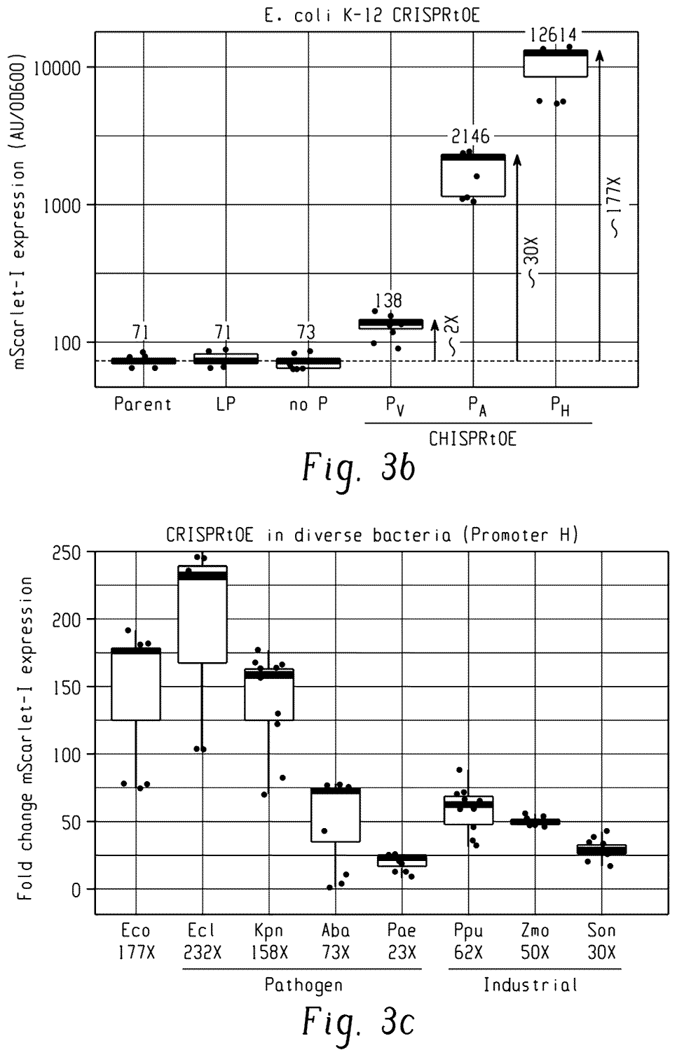

It was next considered whether CRISPRt could be modified into an overexpression system by inserting outward-facing promoters into the Tn6677 transposon that would cause increased transcription of downstream genes (CRISPRtOE (FIG. 3a)). Constitutive promoters of varying strength were inserted into Tn6677 with the goal of creating an OE gradient (data not shown). To quantify the OE activity of CRISPRtOE, a “test” strain was generated with a promoterless mScarlet-I reporter gene integrated into the E. coli K-12 genome. This cassette contained a well-characterized protospacer upstream of mScarlet-I to act as a “landing pad” (LP) for CRISPRtOE transposons (FIG. 3a and data not shown). The test strain showed negligible mScarlet-I expression in the absence of an upstream CRISPRtOE insertion (FIG. 3b).

CRISPRtOE achieved a gradient of mScarlet-I overexpression across our series of tested promoters (FIG. 3b). CRISPRtOE containing the strong P_{H} promoter showed a ˜177-fold increase in mScarlet-I expression, while weaker promoters showed intermediate increases. CRISPRtOE lacking a promoter (no P) failed to stimulate transcription above background, although insertions in the reverse orientation (L-R) were excluded from this analysis due to cryptic promoter activity (data not shown). Small variations in overexpression were observed depending on the precise location of insertion, possibly due to changes in mRNA folding.

CRISPRtOE activity was then demonstrated in diverse bacteria with medical and industrial relevance. The Alphaproteobacterium, Zymomonas mobilis, is a promising biofuel producer and emerging model for bacteria with naturally reduced genomes. To assay CRISPRtOE function in Z. mobilis, an mScarlet-I test strain was generated and expression measured following insertion of CRISPRtOE transposons (data not shown). CRISPRtOE could produce a gradient of overexpression in Z. mobilis up to ˜50-fold. Differences in the fold effects of CRISPRtOE across species likely reflect variation in promoter activity among our constitutive promoter set.

This approach was then expanded to a panel of eight total species including the Gram-negative ESKAPE pathogens Klebsiella pneumoniae, Acinetobacter baumannii, Pseudomonas aeruginosa, and Enterobacter cloacae, as well as the industrially and environmentally relevant strains Pseudomonas putida and Shewanella oneidensis, focusing our strongest promoter (PH) to reduce experimental complexity (FIG. 3c and data not shown). CRISPRtOE could upregulate mScarlet-I expression in all tested species, with an overexpression range of 232-fold (E. cloacae) to 30-fold (S. oneidensis). The CRISPRtOE insertion position in most species was centered at 33 bp upstream of mScarlet-I, although A. baumannii showed a wider range of positions (data not shown). The modular design of CRISPRtOE enables facile swapping of promoter sequences with organism-specific or inducible promoters. CRISPRtOE is an effective overexpression strategy for diverse bacteria.

### Example 4: Pooled CRISPRtOE Recapitulates Known Antibiotic Targets

There is an urgent need to develop new therapeutics to stem the rising tide of antibiotic resistance. One major bottleneck in the process of translating new antimicrobials to the clinic is determining the mode of action (MOA), including identifying the direct target. We previously showed that CRISPRi could be used to determine the direct targets of uncharacterized antibiotics by observing synergy between knockdown strains and sub-lethal concentrations of antibiotic. However, CRISPRtOE could provide an even more straightforward avenue to antibiotic target discovery because strains with overexpressed target proteins would outgrow competitor strains in a pooled context. For instance, it has been shown that OE of folA, encoding the trimethoprim (TMP) target, Dihydrofolate reductase (DHFR), can substantially increase resistance to TMP. Likewise, murA, encoding the fosfomycin (FOS) target, UDP-NAG enolpyruvyl transferase (MurA), can provide FOS resistance when overexpressed.

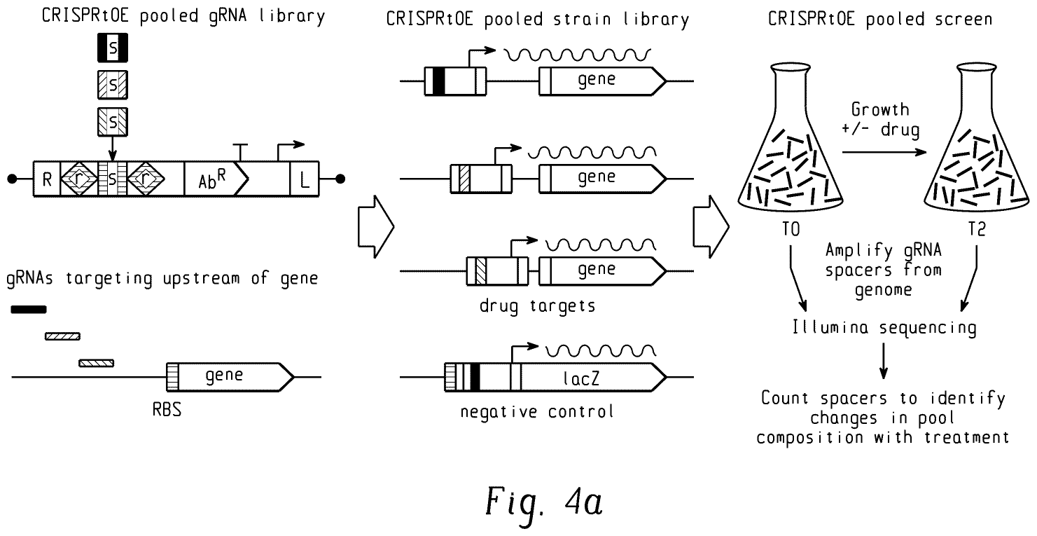

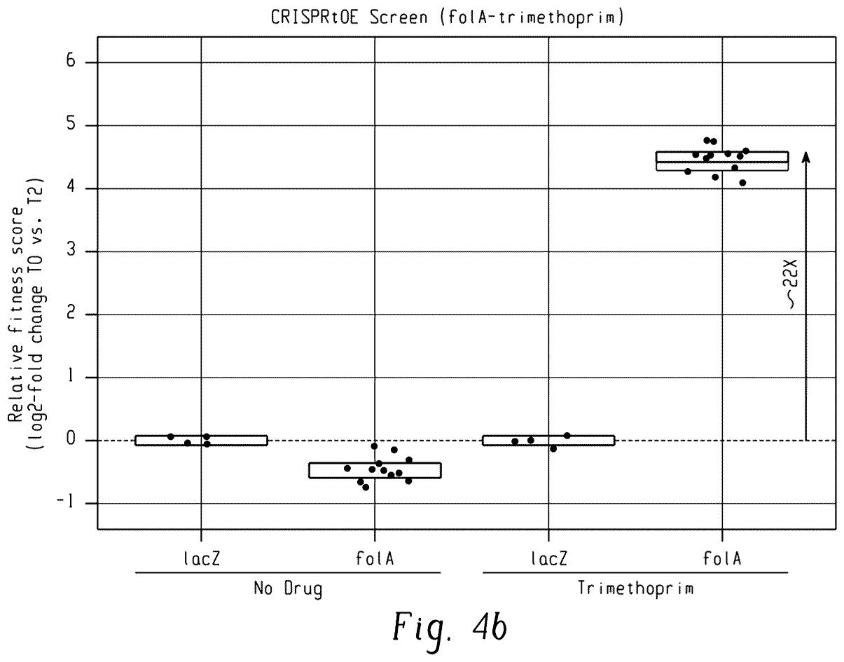

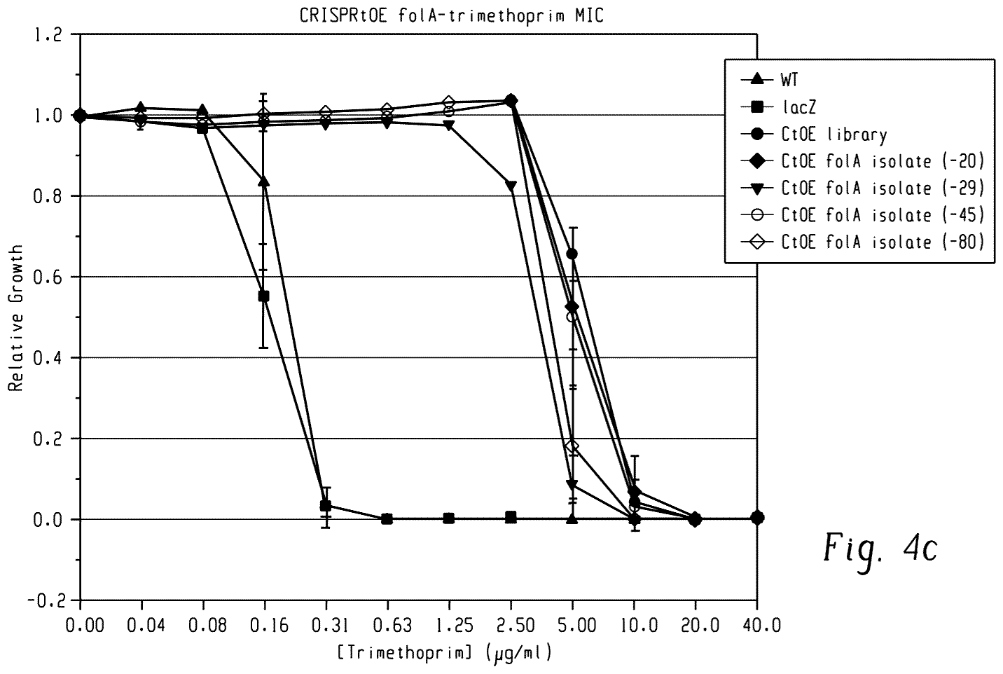

To validate CRISPRtOE as a tool for functional interrogation of endogenous genes and antibiotic target discovery, the folA and murA genes in E. coli K-12 were overexpressed and phenotyped. Because of the anticipated variation in integration efficiency and overexpression efficacy, the region upstream of folA and murA was tiled with 12 unique spacers per gene and a negative control library was created that inserted into the lacZ coding region (FIG. 4a). To test for overexpression phenotypes, the folA or murA library was mixed 50:50 with the lacZ control library and cells were grown in the presence/absence of TMP or FOS, respectively. Strikingly, all CRISPRtOE gRNA spacers targeting folA or murA increased competitive fitness in the context of TMP or FOS, respectively (FIG. 4b). This competitive advantage led to a 22-fold increase in the abundance of folA OE strains and a 52-fold increase in murA OE strains, relative to lacZ controls. To further characterize the impact of CRISPRtOE on TMP and FOS resistance, Minimal Inhibitory Concentration (MIC) strip assays were performed on individual insertion isolates (data not shown). CRISPRtOE strains showed dramatically increased resistance, in some cases with an MIC change that approached the maximum value of the scale. Because FOS showed a high frequency of spontaneous resistant suppressors in MIC strip assays, TMP was selected for subsequent broth microdilution MIC measurements for the wild-type parent strain, a lacZ negative control, and folA CRISPRtOE isolates with insertions at various positions upstream of folA (due to different spacer usage (FIG. 4c)). Substantial shifts in MIC (˜15 to 30-fold) were observed between the parent strains and folA CRISPRtOE isolates, demonstrating that CRISPRtOE phenotypes were robust to insertion distance upstream of the target gene. CRISPRtOE reliably upregulates endogenous target genes and facilitates antibiotic function discovery.

### Example 5: Genome-Scale CRISPRtOE Reveals Antibiotic Targets and Resistance Pathways

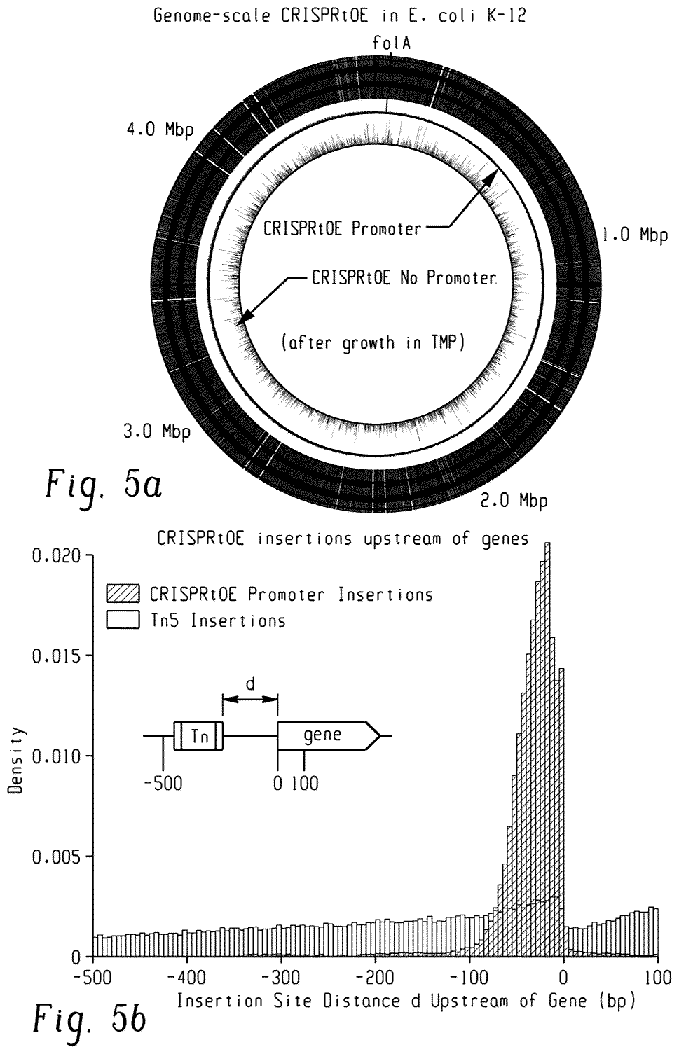

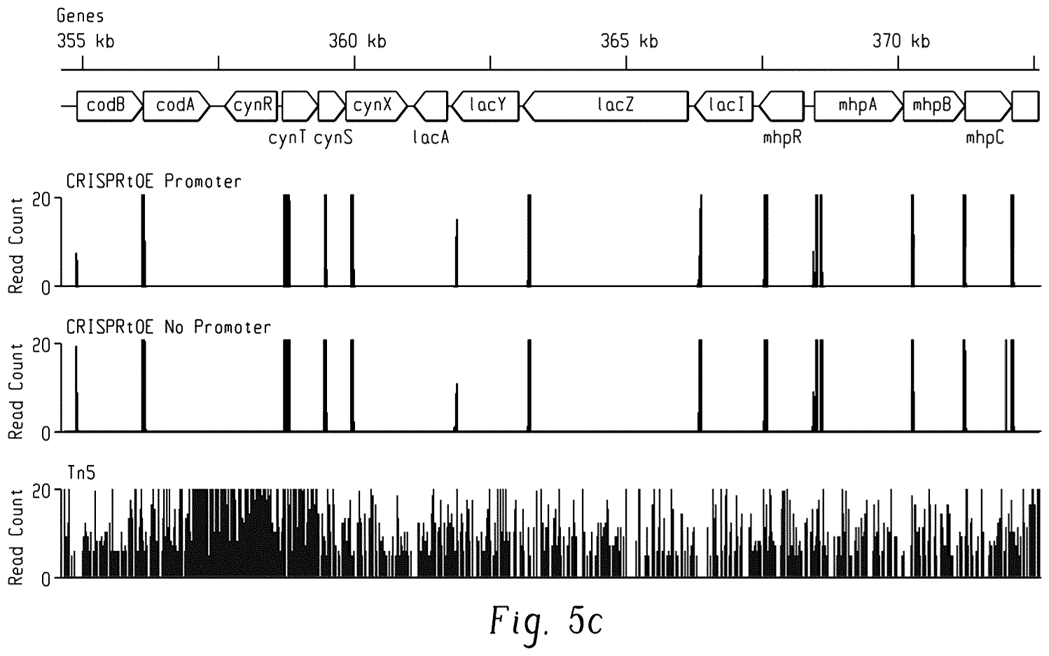

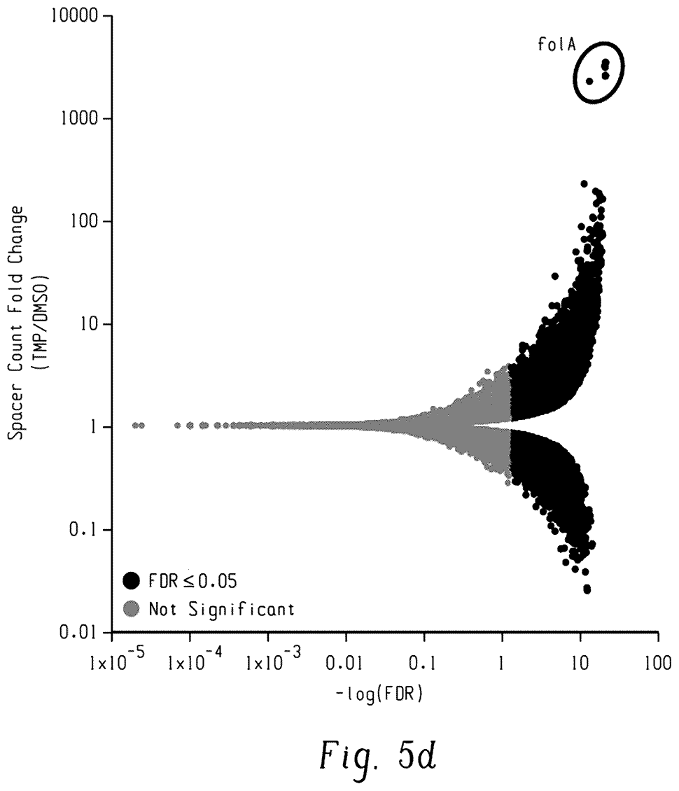

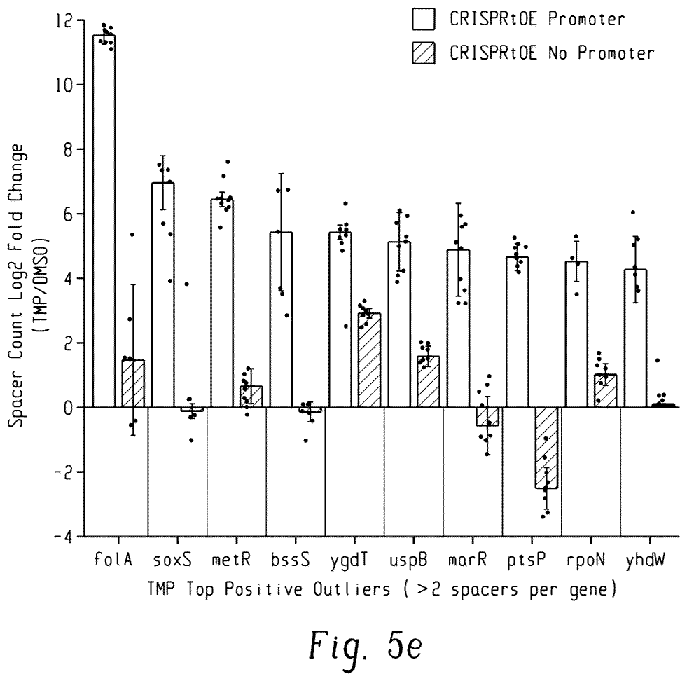

Buoyed by the success with folA and murA, the scope of pooled CRISPRtOE screening was expanded to the genome-scale. All protein coding genes in the E. coli K-12 genome were targeted with ˜10 gRNAs designed to integrate CRISPRtOE transposons within a small window upstream of genes with disrupting downstream coding sequences (45,767 guides total, (FIG. 5)). Further, two pooled libraries were generated, with and without promoter H to distinguish between effects caused by promoter activity versus insertion. CRISPRtOE effectively targeted regions upstream of genes across the genome, without obvious hot spots or depleted regions (FIG. 5a, see “CRISPRtOE No Promoter”). To characterize the insertion of CRISPRtOE transposons relative to genes, the distance between transposon ends and gene starts was calculated, finding a tight distribution of insertions largely within 100 bp of the 5′ ends of genes and centered at ˜20 bp upstream (FIG. 5b). This tight CRISPRtOE distribution showed a stark contrast with the nearly flat distribution of Tn5 insertions, which are known to occur pseudo-randomly. For instance, the distinction between CRISPRtOE targeted and Tn5 pseudo-random insertion was clearly visible at the lac locus (FIG. 5c).

To test the ability of CRISPRtOE to identify antibiotic-relevant gene phenotypes at the genome-scale, the libraries were grown in the presence or absence of a sub-lethal concentration of TMP and relative strain abundance was measured. Biological replicates showed excellent agreement at the gene level (R² values ranged from 0.95 to 0.97), underscoring the high reproducibility of CRISPRtOE. folA-associated spacers were identified as clear positive outliers (>1000-fold enrichment), demonstrating the exquisite sensitivity and specificity of the CRISPRtOE screen in identifying antibiotic targets (FIGS. 5a, 5d, and 5e). Substantial folA enrichment was only seen in the PH-containing library, indicating the CRISPRtOE promoter activity, rather than insertion per se, was responsible for the phenotype (FIG. 5e). This observation could be generalized to the whole genome, as relative strain abundances of the P_{H} and no promoter libraries after TMP treatment were not correlated (R²=0.037). Importantly, data quality was improved by computationally eliminating aberrantly inserted transposons based on distance between the intended target (defined by the spacer sequence) and the insertion position/orientation (defined by Tn-seq). This mostly impacted the “no promoter” data as cryptic transcription from transposons that inserted in the L-R orientation also resulted in overexpression phenotypes (data not shown). The libraries likely contained many more guides than was necessary to uncover phenotypes, as gene-level phenotypes were robust when the number of spacers per genes was reduced from ˜10 to 3 (R²=0.93 data not shown) and considering only guides that targeted the first gene of operons returned many of the same top hits (data not shown).

Other significant outliers revealed potential TMP resistance or susceptibility mechanisms (FIG. 5e and data not shown). One of the most resistant outlier genes was soxS, encoding an AraC-type transcription activator known to regulate genes involved in antibiotic resistance (FIG. 5e). Indeed, gene set enrichment analysis showed that several members of the AraC and MarR families were resistant outliers in our screen, including marR, rob, soxR, and marA (FDR=0.0055, afc test) (FIG. 5e and data not shown), which notably includes genes encoding the AcrA and AcrB components of the AcrAB-TolC multidrug efflux pump. Consistent with this, CRISPRtOE of the acrA gene also increased TMP resistance (data not shown). TMP treatment reduces cellular methionine levels due to the involvement of tetrahydrofolate (the product of DHFR) in methionine biosynthesis. CRISPRtOE of the metR gene, which encodes a positive regulator of methionine biosynthesis genes, increased TMP resistance, possibly by increasing flux through the pathway. In contrast, metF overexpression was highly deleterious in the presence of TMP (data not shown), likely due to depletion of 5,10-methylenetetrahydrofolate pools normally used by the ThyA-DHFR pathway to generate tetrahydrofolate. Interestingly, CRISPRtOE of folM, which is thought to encode a protein with DHFR activity, was highly toxic to TMP-treated cells (data not shown). Although unintuitive, this result is consistent with other genome-scale phenotyping studies that found that disruption of folM increases resistance to TMP. Finally, genes involved in biosynthesis of the Enteric Common Antigen (ECA), a key component of the enterobacterial outer membrane, were functionally enriched among sensitive outliers (FDR=5.58e-05, afc test) (data not shown). Previous work has shown that disruption of the ECA pathway also increases TMP sensitivity, possibly by sequestering thymidine pools and leading to “hyper-acting” version of thymine-less death. This work suggests that disregulation by overexpression, in addition to disruption, can cause ECA-dependent toxicity in TMP-treated cells. Although ECA genes often exist in complex operons that may be disrupted by insertion, phenotypes for some ECA genes (e.g., rffM) were strongly dependent on the presence of P_{H} (data not shown). Taken together, CRISPRtOE is a facile approach for elucidating direct antibiotic targets and related pathways.

### DISCUSSION

Functional genomics approaches that are robustly scalable and readily applicable to diverse bacteria are required to bridge the yawning gap between genome sequencing and gene function assessment. Described herein is a targeted, systematic, and practical approach for genome-scale gene overexpression in bacteria. By modifying Vc CAST for use in functional genomics (FIG. 1) and defining non-transcribed regions of DNA as high-efficiency insertion sites (FIG. 2), targeted and tunable OE was demonstrated in bacteria with medical, industrial, and basic research relevance (FIG. 3). The genome-scale OE screen for targets and modulators of TMP efficacy highlighted the exquisite specificity of CRISPRtOE in defining genes and pathways that underpin antibiotic function (FIGS. 4 and 5). Given the ease of implementing CRISPRtOE in the organisms explored here, this approach will be readily expandable to other microbes and screening conditions.

CRISPRtOE offers advantages over current tools for both basic and applied biology. The targeted aspect of CRISPRtOE can be used to generate focused libraries that limit size (number of gRNA) and scope (number of targeted genes). Moreover, screen hits can be readily pursued for downstream mechanistic analysis by individually cloning gRNAs used in the screen; such follow-ups are not possible with random transposition approaches. Generating sub-libraries from screen hits enables follow up validation at scale. The ability to insert transposons with or without promoters at the same genomic locus will allow researchers to disentangle the effects of Tn insertion versus overexpression, simplifying hit interpretation. Further, combining CRISPRtOE with existing gene perturbation approaches such as CRISPRi could enable screening for overexpression suppressors of essential functions at the genome-scale.

The proof of principle work demonstrating that CRISPRtOE can identify resistance mechanisms in E. coli K-12 holds promise for extending this approach to clinically relevant pathogens with the goal of improving diagnosis and treatment. Importantly, CRISPRtOE action is similar to clinically relevant antibiotic resistance mechanisms, such as IS element transposition and overexpression of downstream resistance genes. CRISPRtOE may also be valuable for optimization of strains for industrial use. Classically, industrial strain optimization has occurred through loss of function or directed evolution experiments, in some cases followed up by plasmid-based overexpression of mutated pathways. CRISPRtOE streamlines this process, particularly by enabling recreation of optimized strain features in different genetic backgrounds by targeted transposition. Finally, CRISPRtOE-optimized strains will likely be more immediately useful in production, as CRISPRi/a strains are subject to mutational inactivation and CRISPRtOE leaves no heterologously expressed cas genes in the recipient strain.

TABLE 2
gRNA Spacer sequences

| | | SEQ |
|---|---|---|
| | | ID |
| Name | Spacer sequence | NO: |
| LZ1 (Petassi, PMID: | caacttaatcgccttgcagcacatcccccttt | 16 |
| 33271061) | | |
| gfp1 | gtggagagggtgaaggtgatgctacaaacgga | 17 |
| oJMP1950-lib-LP1-OE | tgcagcacatccccctttcccaaaggagctga | 18 |
| oJMP1950-lib-LP2-OE | ttgcagcacatccccctttcccaaaggagctg | 19 |
| oJMP1950-lib-LP3-OE | ccttgcagcacatccccctttcccaaaggagc | 20 |
| oJMP1950-lib-LP4-OE | taatcgccttgcagcacatccccctttcccaa | 21 |
| oJMP1950-lib-LP5-OE | aacttaatcgccttgcagcacatccccctttc | 22 |
| (lacZ5) | | |
| oJMP1950-lib-LP6-OE | caacttaatcgccttgcagcacatcccccttt | 23 |
| (lacZ6) | | |
| oJMP1950-lib-LP7-OE | cccaacttaatcgccttgcagcacatccccct | 24 |
| (lacZ7) | | |
| oJMP1950-lib-LP8-OE | acccaacttaatcgccttgcagcacatccccc | 25 |
| (lacZ8) | | |
| oJMP1950-lib-LP9-OE | CCacccaacttaatcgccttgcagcacatccc | 26 |
| oJMP1950-lib-LP10-OE | tCCacccaacttaatcgccttgcagcacatcc | 27 |
| oJMP1950-lib-LP11-OE | CCtCCacccaacttaatcgccttgcagcacat | 28 |
| oJMP1950-lib-LP12-OE | gCCtCCacccaacttaatcgccttgcagcaca | 29 |
| oJMP1950-lib-folA1-OE | gaatataaaattttcctcaacatcatcctcgc | 30 |
| oJMP1950-lib-folA2-OE | gcagaatataaaattttcctcaacatcatcct | 31 |
| oJMP1950-lib-folA3-OE | agcagaatataaaattttcctcaacatcatcc | 32 |
| oJMP1950-lib-folA4-OE | ccagcagaatataaaattttcctcaacatcat | 33 |
| oJMP1950-lib-folA5-OE | cgccagcagaatataaaattttcctcaacatc | 34 |
| oJMP1950-lib-folA6-OE | tggactcgccagcagaatataaaattttcctc | 35 |
| oJMP1950-lib-folA7-OE | gggagagagcgtggactcgccagcagaatata | 36 |
| oJMP1950-lib-folA8-OE | agggagagagcgtggactcgccagcagaatat | 37 |
| oJMP1950-lib-folA9-OE | agtccagggagagagcgtggactcgccagcag | 38 |
| oJMP1950-lib-folA10-OE | gcgagtccagggagagagcgtggactcgccag | 39 |
| oJMP1950-lib-folA11-OE | ttgtaatgcggcgagtccagggagagagcgtg | 40 |
| oJMP1950-lib-folA12-OE | gtttgtttttgtttcattgtaatgcggcgagt | 41 |
| oJMP1950-lib-murA1-OE | tgcggagtgggcgcgcgatcgcaaactgaacg | 42 |
| oJMP1950-lib-murA2-OE | ctgcggagtgggcgcgcgatcgcaaactgaac | 43 |
| oJMP1950-lib-murA3-OE | cctgcggagtgggcgcgcgatcgcaaactgaa | 44 |
| oJMP1950-lib-murA4-OE | tatacccctgcggagtgggcgcgcgatcgcaa | 45 |
| oJMP1950-lib-murA5-OE | aagcgtatacccctgcggagtgggcgcgcgat | 46 |
| oJMP1950-lib-murA6-OE | atcaaagcgtatacccctgcggagtgggcgcg | 47 |
| oJMP1950-lib-murA7-OE | gtgtcgatcaaagcgtatacccctgcggagtg | 48 |
| oJMP1950-lib-murA8-OE | tgctgtgtcgatcaaagcgtatacccctgcgg | 49 |
| oJMP1950-lib-murA9-OE | ttcatgctgtgtcgatcaaagcgtatacccct | 50 |
| oJMP1950-lib-murA10-OE | cattcatgctgtgtcgatcaaagcgtataccc | 51 |
| oJMP1950-lib-murA11-OE | gcattcatgctgtgtcgatcaaagcgtatacc | 52 |
| oJMP1950-lib-murA12-OE | gataaccgcattcatgctgtgtcgatcaaagc | 53 |
| oJMP2005-lib-T01 | aggcagtgatcaaggagttcatgcggttcaag | 54 |
| oJMP2005-lib-T02 | tgcggttcaaggtgcacatggagggctccatg | 55 |
| oJMP2005-lib-T03 | tggagggctccatgaacggccacgagttcgag | 56 |
| oJMP2005-lib-T05 | agggcgagggccgcccctacgagggcacccag | 57 |
| oJMP2005-lib-T06 | tacgagggcacccagaccgccaagctgaaggt | 58 |
| oJMP2005-lib-T07 | cagaccgccaagctgaaggtgaccaagggtgg | 59 |
| oJMP2005-lib-T08 | gaaggtgaccaagggtggccccctgcccttct | 60 |
| oJMP2005-lib-T09 | aagggtggccccctgcccttctcctgggacat | 61 |
| oJMP2005-lib-T10 | ctgcccttctcctgggacatcctgtcccctca | 62 |
| oJMP2005-lib-T11 | tcctgtcccctcagttcatgtacggctccagg | 63 |
| oJMP2005-lib-T12 | gctccagggccttcaTcaagcaccccgccgac | 64 |
| oJMP2005-lib-T13 | agcaccccgccgacatccccgactactataag | 65 |
| oJMP2005-lib-T14 | cgccgacatccccgactactataagcagtcct | 66 |
| oJMP2005-lib-T15 | actataagcagtccttccccgagggcttcaag | 67 |
| oJMP2005-lib-T16 | gagggcttcaagtgggagcgcgtgatgaactt | 68 |
| oJMP2005-lib-T17 | cgtgatgaacttcgaggacggcggcgccgtga | 69 |
| oJMP2005-lib-T18 | gcggcgccgtgaccgtgacccaggacacctcc | 70 |
| oJMP2005-lib-T19 | ggacacctccctggaggacggcaccctgatct | 71 |
| oJMP2005-lib-T20 | gcaccctgatctacaaggtgaagctccgcggc | 72 |
| oJMP2005-lib-T21 | aggtgaagctccgcggcaccaacttccctcct | 73 |
| oJMP2005-lib-T22 | aacttccctcctgacggccccgtaatgcagaa | 74 |
| oJMP2005-lib-T23 | gtaatgcagaagaaAacaatgggctgggaagc | 75 |
| oJMP2005-lib-T24 | atgggctgggaagcgtccaccgagcggttgta | 76 |
| oJMP2005-lib-B01 | gcatgaactCCttgatcactgcctcgcccttg | 77 |
| oJMP2005-lib-B02 | ccatgtgcaccttgaaccgcatgaactCCttg | 78 |
| oJMP2005-lib-B03 | tggccgttcatggagccctccatgtgcacctt | 79 |
| oJMP2005-lib-B04 | ctcgatctcgaactcgtggccgttcatggagc | 80 |
| oJMP2005-lib-B06 | cgtaggggcggccctcgccctcgccctcgatc | 81 |
| oJMP2005-lib-B07 | gcttggcggtctgggtgccctcgtaggggcgg | 82 |
| oJMP2005-lib-B08 | ttggtcaccttcagcttggcggtctgggtgcc | 83 |
| oJMP2005-lib-B09 | gggggccacccttggtcaccttcagcttggcg | 84 |
| oJMP2005-lib-B10 | aggagaagggcagggggccacccttggtcacc | 85 |
| oJMP2005-lib-B11 | gaggggacaggatgtcccaggagaagggcagg | 86 |
| oJMP2005-lib-B12 | tggagccgtacatgaactgaggggacaggatg | 87 |
| oJMP2005-lib-B13 | gggtgcttgAtgaaggccctggagccgtacat | 88 |
| oJMP2005-lib-B14 | tatagtagtcggggatgtcggcggggtgcttg | 89 |
| oJMP2005-lib-B15 | ctcggggaaggactgcttatagtagtcgggga | 90 |
| oJMP2005-lib-B16 | acttgaagccctcggggaaggactgcttatag | 91 |
| oJMP2005-lib-B17 | cgcgctcccacttgaagccctcggggaaggac | 92 |
| oJMP2005-lib-B18 | gcgccgccgtcctcgaagttcatcacgcgctc | 93 |
| oJMP2005-lib-B19 | tgggtcacggtcacggcgccgccgtcctcgaa | 94 |
| oJMP2005-lib-B20 | gtcctccagggaggtgtcctgggtcacggtca | 95 |
| oJMP2005-lib-B21 | tgtagatcagggtgccgtcctccagggaggtg | 96 |
| oJMP2005-lib-B22 | gcggagcttcaccttgtagatcagggtgccgt | 97 |
| oJMP2005-lib-B23 | ggagggaagttggtgccgcggagcttcacctt | 98 |
| oJMP2005-lib-B24 | tctgcattacggggccgtcaggagggaagttg | 99 |

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context. directed by the gRNA.

1. A system for RNA-guided DNA transposition in a bacterial cell's genome, the system comprising
a) a first non-replicating plasmid expressing an engineered Clustered Regularly Interspaced Short Palindromic Repeats associated (Cas) (CRISPR-Cas) protein and a transposase; and
b) a second non-replicating donor plasmid, the second non-replicating donor plasmid comprising an antibiotic selection marker, a guide RNA (gRNA) sequence and a cargo sequence flanked by a right transposon end sequence and a left transposon end sequence, wherein the gRNA is specific for a target site, and wherein the cargo sequence comprises a promoter, a protein expression sequence, or a combination thereof,
wherein the first non-replicating plasmid and the second non-replicating donor plasmid, when introduced into a cell, provide insertion of the cargo sequence into the bacterial cell's genome as directed by the gRNA.

2. The system of claim 1, wherein the engineered CRISPR-Cas system comprises Cas5, Cas6, Cas7 and Cas8; or a Class 2 CRISPR-Cas system.

3. The system of claim 2, wherein the engineered CRISPR-Cas system comprises a Type I-F variant where the Cas8 and Cas5 form a Cas8-Cas5 fusion.

4. The system of claim 1, wherein the transposase is a Tn7 transposon comprising i) Transposon 7 protein A (TnsA) TnsA, ii) Transposon 7 protein B (Tns B), iii) Transposon 7 protein C (Tns C), and iv) transposition of integron protein Q (TniQ), and wherein the transposase is derived from Vibrio cholerae Tn6677.

5. The system of claim 4, wherein each of said right and left transposon end sequences comprises at least one TnsB binding site.

6. The system of claim 1, wherein the transposase is a Mu transposase.

7. The system of claim 1, Mu transposase comprises MuA, MuB, MuC, or a combination thereof.

8. The system of claim 1, wherein the gRNA binds the transcribed strand of the target site.

9. The system of claim 1, wherein the bacteria comprises Zymomonas mobilis, Klebsiella pneumoniae, Acinetobacter baumannii, Pseudomonas aeruginosa, Enterobacter cloacae, Pseudomonas putida, Shewanella oneidensis, or E. coli.

10. The system of claim 1, wherein the cargo sequence comprises a promoter.

11. The system of claim 1, comprising a pool of second non-replicating donor plasmids, wherein each member of the pool comprises the antibiotic selection marker, a member guide RNA (gRNA) sequence and a member cargo sequence flanked by the right transposon end sequence and the left transposon end sequence, wherein each member gRNA is specific for a target site, and wherein each member cargo sequence comprises a promoter, a protein expression sequence, or a combination thereof.

12. The system of claim 11, wherein the member guide RNA sequences are specific for a single target site, or a plurality of target sites.

13. The system of claim 11, wherein the member cargo sequences are a plurality of promoters.

14. The system of claim 13, wherein the member guide RNA sequences are specific for a plurality of target sites upstream of a targeted gene, and wherein at least a portion of the promoters provide overexpression of the targeted gene.

15. The system of claim 13, wherein the targeted gene is a protein coding gene.

16. The system of claim 15, wherein the protein coding gene is involved in the mechanism of action of an antibiotic or provides resistance to growth inhibition during biofuel production.

17. A method of RNA-guided DNA transposition in a bacterial cell's genome, comprising transferring the first and second non-replicating plasmids of claim 1 into the bacterial cell, providing insertion of the cargo sequence into the bacterial cell's genome as directed by the gRNA.


SEQUENCE LISTING (99 sequences)

SEQ ID NO: 1 - protein, 640 aa, Vibrio cholerae
MQTLKELIASNPDDLTTELKRAFRPLTPHIAIDGNELDALTILVNLTDKTDDQKDLLDRAKCKQKLRDEKWWASCINCVNYRQSHNPKFPDIRSEGVIRTQALGELPSFLLSSSKIPPYHWSYSHDSKYVNKSAFLTNEFCWDGEISCLGELLKDADHPLWNTLKKLGCSQKTCKAMAKQLADITLTTINVTLAPNYLTQISLPDSDTSYISLSPVASLSMQSHFHQRLQDENRHSAITRFSRTTNMGVTAMTCGGAFRMLKSGAKFSSPPHHRLNSKRSWLTSEHVQSLKQYQRLNKSLIPENSRIALRRKYKIELQNMVRSWFAMQDHTLDSNILIQHLNHDLSYLGATKRFAYDPAMTKLFTELLKRELSNSINNGEQHTNGSFLVLPNIRVCGATALSSPVTVGIPSLTAFFGFVHAFERNINRTTSSFRVESFAICVHQLHVEKRGLTAEFVEKGDGTISAPATRDDWQCDVVFSLILNTNFAQHIDQDTLVTSLPKRLARGSAKIAIDDFKHINSFSTLETAIESLPIEAGRWLSLYAQSNNNLSDLLAAMTEDHQLMASCVGYHLLEEPKDKPNSLRGYKHAIAECIIGLINSITFSSETDPNTIFWSLKNYQNYLVVQPRSINDETTDKSSL

SEQ ID NO: 2 - protein, 352 aa, Vibrio cholerae
MKLPTNLAYERSIDPSDVCFFVVWPDDRKTPLTYNSRTLLGQMEAASLAYDVSGQPIKSATAEALAQGNPHQVDFCHVPYGASHIECSFSVSFSSELRQPYKCNSSKVKQTLVQLVELYETKIGWTELATRYLMNICNGKWLWKNTRKAYCWNIVLTPWPWNGEKVGFEDIRTNYTSRQDFKNNKNWSAIVEMIKTAFSSTDGLAIFEVRATLHLPTNAMVRPSQVFTEKESGSKSKSKTQNSRVFQSTTIDGERSPILGAFKTGAAIATIDDWYPEATEPLRVGRFGVHREDVTCYRHPSTGKDFFSILQQAEHYIEVLSANKTPAQETINDMHFLMANLIKGGMFQHKGD

SEQ ID NO: 3 - protein, 199 aa, Vibrio cholerae
MKWYYKTITFLPELCNNESLAAKCLRVLHGFNYQYETRNIGVSFPLWCDATVGKKISFVSKNKIELDLLLKQHYFVQMEQLQYFHISNTVLVPEDCTYVSFRRCQSIDKLTAAGLARKIRRLEKRALSRGEQFDPSSFAQKEHTAIAHYHSLGESSKQTNRNFRLNIRMLSEQPREGNSIFSSYGLSNSENSFQPVPLI

SEQ ID NO: 4 - protein, 229 aa, Vibrio sp.
MTSLPTPSAITTSALEYAFHTPARNLTKSRGKNIHRYVSVKMSKRITVESTLECDACYHFDFEPSIVRFCAQPIRFLYYLNGQSHSYVPDFLVQFDTNEFVLYEVKSAYAKNKPDFDVEWEAKVKAATELGLELELVEESDIRDTVVLNNLKRMHRYASKDELNNVHNSLLKIIKYNGAQSARCLGEQLGLKGRTVLPILCDLLSRCLLDTRLDKPLSLESRFELASYG

SEQ ID NO: 5 - protein, 603 aa, Vibrio sp.
MAKKGFSSFHRKAVSSQDTLESIELVSSANCLESVTYQDISAFPETIAVEINFRLSILRFLARKCETIVAKSIEPHRVELQQNYSRKIPSAITIYRWWLAFRKSDYNPISLAPNIKDRGNRETKVSTVVDSIMEQAVERVISGRKVNVSSAYKRVRRKVRQYNLTHGTKYTYPKYESVRKRVKKKTPFELLAAGKGERVAKREFRRMGKKILTSSVLERVEIDHTVVDLFAVHEEYRIPLGRPWLTQLVDCYSKAVIGFYLGFEPPSYVSVSLALKNAIQRKDDLISSYESIENEWLCYGIPDLLVTDNGKEFLSKAFDQACESLLINVHQNKVETPDNKPHVERNYGTINTSLLDDLPGKSFSQYLQREGYDSVGEATLTLNEIREIYLIWLVDIYHKKPNQRGTNCPNVAWKKGCQEWEPEEFSGSKDELDFKFAIVDYKQLTKVGITVYKELSYSNDRLAEYRGKKGNHKVQFKYNPECMAVIWVLDEDMNEYFTVNAIDYEYASRVSLWQHKYNMKYQAELNSAEYDEDKEIDAEIKIEEIADRSIVKTNKIRARRRGARHQENSARAKSISNANPASIQKHEDEIVSADNDDWDIDYV

SEQ ID NO: 6 - protein, 330 aa, Vibrio sp.
MSETREARISRAKRAFVSTPSVRKILSYMDRCRDLSDLESEPTCMMVYGASGVGKTTVIKKYLNQNRRESEAGGDIIPVLHIELPDNAKPVDAARELLVEMGDPLALYETDLARLTKRLTELIPAVGVKLIIIDEFQHLVEERSNRVLTQVGNWLKMILNKTKCPIVIFGMPYSKVVLQANSQLHGRFSIQVELRPFSYQGGRGVFKTFLEYLDKALPFEKQAGLANESLQKKLYAFSQGNMRSLRNLIYQASIEAIDNQHETITEEDFVFASKLTSGDKPNSWKNPFEEGVEVTEDMLRPPPKDIGWEDYLRHSTPRVSKPGRNKNFFE

SEQ ID NO: 7 - protein, 669 aa, Haemophilus influenzae
MKNWVTAKEIAGIGGLSKHPTNVNRLARKEKWIFREIQGVQGGGYEYSFSSLPLEVQTEYLLKHSEELKVNKENNDSNQQTMIESAWNVLASATFEQEKRAERRFQAVVKVARLVENKIPLMKAFEQVVALYATDSNDETISKGSLKRWWYKVKTHPQGIWLPLLLDRTERDNSCRWADISDKAWAFFCADYLRKSKPKFSVCYYRLTLAAEENDWTIPSLSSLKRKFYNEFTEARNRISRWGEHELRELTAPQIRTVMDLEAYEIVNGDGYQHNVFVDWYEDGRPPIRPKTWFWQDVRTRRILSYCVDDSENGDQIRQATLRMIKQYGIPKTILMDNTRAASDKQTTQQRKRGKRQTAGIKIDGMFDRLGIKVIRTLVFKGRGNGRAKPIERAFKRDSLPAYVDGDARLEAFFTGWSVTEKTEDYQFKKGVNKALFLEILEQGVRLWNDKAGRETELGQGIFSADQLWARDYAQTRQTFATEEQLRQLMMLGESTKVDKHGRFTLKAGYVLNNQKNVYEAQALIGGNVGSVIVRYDPDDLHGTVHIYDQNGVYLCNGECVEKIAFNSEEGARVQKRLINEQRRITKKMVDNHEKLSEHEMAQYRKQFEQSAADFVEPAEKQVFSWTQFADGNTVKTIKLDHELETETEEQNTLSFEQDLFNGLKQVQK

SEQ ID NO: 8-15 - skipped (8 entries)

SEQ ID NO: 16 - DNA, 32 bp, synthetic construct
  source 1..32: mol_type = other DNA
caacttaatcgccttgcagcacatcccccttt

SEQ ID NO: 17 - DNA, 32 bp, synthetic construct
  source 1..32: mol_type = other DNA
gtggagagggtgaaggtgatgctacaaacgga

SEQ ID NO: 18 - DNA, 32 bp, synthetic construct
  source 1..32: mol_type = other DNA
tgcagcacatccccctttcccaaaggagctga

SEQ ID NO: 19 - DNA, 32 bp, synthetic construct
  source 1..32: mol_type = other DNA
ttgcagcacatccccctttcccaaaggagctg

SEQ ID NO: 20 - DNA, 32 bp, synthetic construct
  source 1..32: mol_type = other DNA
ccttgcagcacatccccctttcccaaaggagc

SEQ ID NO: 21 - DNA, 32 bp, synthetic construct
  source 1..32: mol_type = other DNA
taatcgccttgcagcacatccccctttcccaa

SEQ ID NO: 22 - DNA, 32 bp, synthetic construct
  source 1..32: mol_type = other DNA
aacttaatcgccttgcagcacatccccctttc

SEQ ID NO: 23 - DNA, 32 bp, synthetic construct; identical to SEQ ID NO: 16
  source 1..32: mol_type = other DNA

SEQ ID NO: 24 - DNA, 32 bp, synthetic construct
  source 1..32: mol_type = other DNA
cccaacttaatcgccttgcagcacatccccct

SEQ ID NO: 25 - DNA, 32 bp, synthetic construct
  source 1..32: mol_type = other DNA
acccaacttaatcgccttgcagcacatccccc

SEQ ID NO: 26 - DNA, 32 bp, synthetic construct
  source 1..32: mol_type = other DNA
ccacccaacttaatcgccttgcagcacatccc

SEQ ID NO: 27 - DNA, 32 bp, synthetic construct
  source 1..32: mol_type = other DNA
tccacccaacttaatcgccttgcagcacatcc

SEQ ID NO: 28 - DNA, 32 bp, synthetic construct
  source 1..32: mol_type = other DNA
cctccacccaacttaatcgccttgcagcacat

SEQ ID NO: 29 - DNA, 32 bp, synthetic construct
  source 1..32: mol_type = other DNA
gcctccacccaacttaatcgccttgcagcaca

SEQ ID NO: 30 - DNA, 32 bp, synthetic construct
  source 1..32: mol_type = other DNA
gaatataaaattttcctcaacatcatcctcgc

SEQ ID NO: 31 - DNA, 32 bp, synthetic construct
  source 1..32: mol_type = other DNA
gcagaatataaaattttcctcaacatcatcct

SEQ ID NO: 32 - DNA, 32 bp, synthetic construct
  source 1..32: mol_type = other DNA
agcagaatataaaattttcctcaacatcatcc

SEQ ID NO: 33 - DNA, 32 bp, synthetic construct
  source 1..32: mol_type = other DNA
ccagcagaatataaaattttcctcaacatcat

SEQ ID NO: 34 - DNA, 32 bp, synthetic construct
  source 1..32: mol_type = other DNA
cgccagcagaatataaaattttcctcaacatc

SEQ ID NO: 35 - DNA, 32 bp, synthetic construct
  source 1..32: mol_type = other DNA
tggactcgccagcagaatataaaattttcctc

SEQ ID NO: 36 - DNA, 32 bp, synthetic construct
  source 1..32: mol_type = other DNA
gggagagagcgtggactcgccagcagaatata

SEQ ID NO: 37 - DNA, 32 bp, synthetic construct
  source 1..32: mol_type = other DNA
agggagagagcgtggactcgccagcagaatat

SEQ ID NO: 38 - DNA, 32 bp, synthetic construct
  source 1..32: mol_type = other DNA
agtccagggagagagcgtggactcgccagcag

SEQ ID NO: 39 - DNA, 32 bp, synthetic construct
  source 1..32: mol_type = other DNA
gcgagtccagggagagagcgtggactcgccag

SEQ ID NO: 40 - DNA, 32 bp, synthetic construct
  source 1..32: mol_type = other DNA
ttgtaatgcggcgagtccagggagagagcgtg

SEQ ID NO: 41 - DNA, 32 bp, synthetic construct
  source 1..32: mol_type = other DNA
gtttgtttttgtttcattgtaatgcggcgagt

SEQ ID NO: 42 - DNA, 32 bp, synthetic construct
  source 1..32: mol_type = other DNA
tgcggagtgggcgcgcgatcgcaaactgaacg

SEQ ID NO: 43 - DNA, 32 bp, synthetic construct
  source 1..32: mol_type = other DNA
ctgcggagtgggcgcgcgatcgcaaactgaac

SEQ ID NO: 44 - DNA, 32 bp, synthetic construct
  source 1..32: mol_type = other DNA
cctgcggagtgggcgcgcgatcgcaaactgaa

SEQ ID NO: 45 - DNA, 32 bp, synthetic construct
  source 1..32: mol_type = other DNA
tatacccctgcggagtgggcgcgcgatcgcaa

SEQ ID NO: 46 - DNA, 32 bp, synthetic construct
  source 1..32: mol_type = other DNA
aagcgtatacccctgcggagtgggcgcgcgat

SEQ ID NO: 47 - DNA, 32 bp, synthetic construct
  source 1..32: mol_type = other DNA
atcaaagcgtatacccctgcggagtgggcgcg

SEQ ID NO: 48 - DNA, 32 bp, synthetic construct
  source 1..32: mol_type = other DNA
gtgtcgatcaaagcgtatacccctgcggagtg

SEQ ID NO: 49 - DNA, 32 bp, synthetic construct
  source 1..32: mol_type = other DNA
tgctgtgtcgatcaaagcgtatacccctgcgg

SEQ ID NO: 50 - DNA, 32 bp, synthetic construct
  source 1..32: mol_type = other DNA
ttcatgctgtgtcgatcaaagcgtatacccct

SEQ ID NO: 51 - DNA, 32 bp, synthetic construct
  source 1..32: mol_type = other DNA
cattcatgctgtgtcgatcaaagcgtataccc

SEQ ID NO: 52 - DNA, 32 bp, synthetic construct
  source 1..32: mol_type = other DNA
gcattcatgctgtgtcgatcaaagcgtatacc

SEQ ID NO: 53 - DNA, 32 bp, synthetic construct
  source 1..32: mol_type = other DNA
gataaccgcattcatgctgtgtcgatcaaagc

SEQ ID NO: 54 - DNA, 32 bp, synthetic construct
  source 1..32: mol_type = other DNA
aggcagtgatcaaggagttcatgcggttcaag

SEQ ID NO: 55 - DNA, 32 bp, synthetic construct
  source 1..32: mol_type = other DNA
tgcggttcaaggtgcacatggagggctccatg

SEQ ID NO: 56 - DNA, 32 bp, synthetic construct
  source 1..32: mol_type = other DNA
tggagggctccatgaacggccacgagttcgag

SEQ ID NO: 57 - DNA, 32 bp, synthetic construct
  source 1..32: mol_type = other DNA
agggcgagggccgcccctacgagggcacccag

SEQ ID NO: 58 - DNA, 32 bp, synthetic construct
  source 1..32: mol_type = other DNA
tacgagggcacccagaccgccaagctgaaggt

SEQ ID NO: 59 - DNA, 32 bp, synthetic construct
  source 1..32: mol_type = other DNA
cagaccgccaagctgaaggtgaccaagggtgg

SEQ ID NO: 60 - DNA, 32 bp, synthetic construct
  source 1..32: mol_type = other DNA
gaaggtgaccaagggtggccccctgcccttct

SEQ ID NO: 61 - DNA, 32 bp, synthetic construct
  source 1..32: mol_type = other DNA
aagggtggccccctgcccttctcctgggacat

SEQ ID NO: 62 - DNA, 32 bp, synthetic construct
  source 1..32: mol_type = other DNA
ctgcccttctcctgggacatcctgtcccctca

SEQ ID NO: 63 - DNA, 32 bp, synthetic construct
  source 1..32: mol_type = other DNA
tcctgtcccctcagttcatgtacggctccagg

SEQ ID NO: 64 - DNA, 32 bp, synthetic construct
  source 1..32: mol_type = other DNA
gctccagggccttcatcaagcaccccgccgac

SEQ ID NO: 65 - DNA, 32 bp, synthetic construct
  source 1..32: mol_type = other DNA
agcaccccgccgacatccccgactactataag

SEQ ID NO: 66 - DNA, 32 bp, synthetic construct
  source 1..32: mol_type = other DNA
cgccgacatccccgactactataagcagtcct

SEQ ID NO: 67 - DNA, 32 bp, synthetic construct
  source 1..32: mol_type = other DNA
actataagcagtccttccccgagggcttcaag

SEQ ID NO: 68 - DNA, 32 bp, synthetic construct
  source 1..32: mol_type = other DNA
gagggcttcaagtgggagcgcgtgatgaactt

SEQ ID NO: 69 - DNA, 32 bp, synthetic construct
  source 1..32: mol_type = other DNA
cgtgatgaacttcgaggacggcggcgccgtga

SEQ ID NO: 70 - DNA, 32 bp, synthetic construct
  source 1..32: mol_type = other DNA
gcggcgccgtgaccgtgacccaggacacctcc

SEQ ID NO: 71 - DNA, 32 bp, synthetic construct
  source 1..32: mol_type = other DNA
ggacacctccctggaggacggcaccctgatct

SEQ ID NO: 72 - DNA, 32 bp, synthetic construct
  source 1..32: mol_type = other DNA
gcaccctgatctacaaggtgaagctccgcggc

SEQ ID NO: 73 - DNA, 32 bp, synthetic construct
  source 1..32: mol_type = other DNA
aggtgaagctccgcggcaccaacttccctcct

SEQ ID NO: 74 - DNA, 32 bp, synthetic construct
  source 1..32: mol_type = other DNA
aacttccctcctgacggccccgtaatgcagaa

SEQ ID NO: 75 - DNA, 32 bp, synthetic construct
  source 1..32: mol_type = other DNA
gtaatgcagaagaaaacaatgggctgggaagc

SEQ ID NO: 76 - DNA, 32 bp, synthetic construct
  source 1..32: mol_type = other DNA
atgggctgggaagcgtccaccgagcggttgta

SEQ ID NO: 77 - DNA, 32 bp, synthetic construct
  source 1..32: mol_type = other DNA
gcatgaactccttgatcactgcctcgcccttg

SEQ ID NO: 78 - DNA, 32 bp, synthetic construct
  source 1..32: mol_type = other DNA
ccatgtgcaccttgaaccgcatgaactccttg

SEQ ID NO: 79 - DNA, 32 bp, synthetic construct
  source 1..32: mol_type = other DNA
tggccgttcatggagccctccatgtgcacctt

SEQ ID NO: 80 - DNA, 32 bp, synthetic construct
  source 1..32: mol_type = other DNA
ctcgatctcgaactcgtggccgttcatggagc

SEQ ID NO: 81 - DNA, 32 bp, synthetic construct
  source 1..32: mol_type = other DNA
cgtaggggcggccctcgccctcgccctcgatc

SEQ ID NO: 82 - DNA, 32 bp, synthetic construct
  source 1..32: mol_type = other DNA
gcttggcggtctgggtgccctcgtaggggcgg

SEQ ID NO: 83 - DNA, 32 bp, synthetic construct
  source 1..32: mol_type = other DNA
ttggtcaccttcagcttggcggtctgggtgcc

SEQ ID NO: 84 - DNA, 32 bp, synthetic construct
  source 1..32: mol_type = other DNA
gggggccacccttggtcaccttcagcttggcg

SEQ ID NO: 85 - DNA, 32 bp, synthetic construct
  source 1..32: mol_type = other DNA
aggagaagggcagggggccacccttggtcacc

SEQ ID NO: 86 - DNA, 32 bp, synthetic construct
  source 1..32: mol_type = other DNA
gaggggacaggatgtcccaggagaagggcagg

SEQ ID NO: 87 - DNA, 32 bp, synthetic construct
  source 1..32: mol_type = other DNA
tggagccgtacatgaactgaggggacaggatg

SEQ ID NO: 88 - DNA, 32 bp, synthetic construct
  source 1..32: mol_type = other DNA
gggtgcttgatgaaggccctggagccgtacat

SEQ ID NO: 89 - DNA, 32 bp, synthetic construct
  source 1..32: mol_type = other DNA
tatagtagtcggggatgtcggcggggtgcttg

SEQ ID NO: 90 - DNA, 32 bp, synthetic construct
  source 1..32: mol_type = other DNA
ctcggggaaggactgcttatagtagtcgggga

SEQ ID NO: 91 - DNA, 32 bp, synthetic construct
  source 1..32: mol_type = other DNA
acttgaagccctcggggaaggactgcttatag

SEQ ID NO: 92 - DNA, 32 bp, synthetic construct
  source 1..32: mol_type = other DNA
cgcgctcccacttgaagccctcggggaaggac

SEQ ID NO: 93 - DNA, 32 bp, synthetic construct
  source 1..32: mol_type = other DNA
gcgccgccgtcctcgaagttcatcacgcgctc

SEQ ID NO: 94 - DNA, 32 bp, synthetic construct
  source 1..32: mol_type = other DNA
tgggtcacggtcacggcgccgccgtcctcgaa

SEQ ID NO: 95 - DNA, 32 bp, synthetic construct
  source 1..32: mol_type = other DNA
gtcctccagggaggtgtcctgggtcacggtca

SEQ ID NO: 96 - DNA, 32 bp, synthetic construct
  source 1..32: mol_type = other DNA
tgtagatcagggtgccgtcctccagggaggtg

SEQ ID NO: 97 - DNA, 32 bp, synthetic construct
  source 1..32: mol_type = other DNA
gcggagcttcaccttgtagatcagggtgccgt

SEQ ID NO: 98 - DNA, 32 bp, synthetic construct
  source 1..32: mol_type = other DNA
ggagggaagttggtgccgcggagcttcacctt

SEQ ID NO: 99 - DNA, 32 bp, synthetic construct
  source 1..32: mol_type = other DNA
tctgcattacggggccgtcaggagggaagttg